(19) United States
(12) Patent Application Publication
(10) Pub. No.: US 2015/0217131 A1
(43) Pub. Date: Aug. 6, 2015

(54) TERAHERTZ PULSE RADIATION IN TREATING SKIN DISORDERS

(71) Applicants: The Governors of the University of Alberta, Edmonton (CA); The University of Lethbridge, Lethbridge (CA)

(72) Inventors: Lyubov Titova, Edmonton (CA); Frank Hegmann, Edmonton (CA); Olga Kovalchuk, Lethbridge, (CA)

(21) Appl. No.: 14/615,153

(22) Filed: Feb. 5, 2015

Related U.S. Application Data
(60) Provisional application No. 61/936,627, filed on Feb. 6, 2014.

(51) Int. Cl. A61N 5/06

(52) U.S. Cl. CPC A61N 5/0616; A61N 2005/066

(57) ABSTRACT

The disclosure provides methods for the treatment of skin disorders through the use of minimally invasive terahertz radiation. The method includes exposing skin cells to terahertz radiation in amount sufficient to modulate gene expression in the skin cells. The modulation of gene expression then results in a reduction of the disease state or aspects thereof in the exposed skin cells.

### RELATED APPLICATIONS

This application claims priority under 35 U.S.C. §119(e) to U.S. Provisional Patent Application No. 61/936,627, filed Feb. 6, 2014, which is incorporated herein by reference.

### BACKGROUND OF THE INVENTION

Non-melanoma skin cancer (NMSC) and psoriasis are two skin conditions that affect a large number of individuals each year. In the United States alone, there are over 3.5 million cases of NMSC while 1-3% of the population of the United States will develop psoriasis in their lifetime.

NMSC can be primarily divided into two types of skin cancers: squamous cell carcinomas and basal cell carcinomas. Basal cell carcinomas account for 80% of all NMSC cases. This type of cancer develops in cells in the lower epidermis. Such carcinomas occur most frequently on the face, ears, neck, scalp, shoulders, and back. Basal cell carcinoma is usually slow growing and rarely metastasizes, but it can cause significant local destruction and disfigurement if neglected or treated inadequately.

The remaining 20% of NMSC is squamous cell carcinoma. Squamous cell carcinoma develops in the upper most layers of the epidermis. Of the over 700,000 new cases of squamous cell carcinomas diagnosed each year in the United States, over 2,500 will result in death.

Psoriasis is a chronic immune mediated skin disorder effecting 1-3% of the population of the United States. Psoriasis is characterized by hyperplasia of keratinocytes resulting in thickening of the epidermis and the presence of red scaly plaques. The lesions in this chronic disease typically are subject to remissions and exacerbations. Some individuals with psoriasis can also develop psoriatic arthritis and joint pain. Moreover, psoriasis can also be a psychological burden on those afflicted by the disease when the affected skin areas are visible to others.

Current treatment of NMSC generally involves surgical excision of the tumor together with a margin of normal tissue and, when surgery is not feasible or desirable, destruction of the tumor cells by ionizing radiation or other means. Treatment for psoriasis involves topical applications, systemic treatments (e.g., drug injections), and phototherapy involving exposure to UV radiation.

Although each method of treatment can be relatively effective, each has its drawback. For example, removal of a NMSC patch can leave a visible scar. On the other hand, not only is moderate to severe psoriasis resistant to topical treatments, but because of its chronic and recurrent nature, systemic therapy or radiation is often required.

Therefore, there is a need for further methods of treating skin disorders that are rapid, minimally invasive, specific for skin, and easy to use.

### SUMMARY

The disclosure provides for a method of using terahertz radiation as a means to modulate gene expression in skin cells having an active skin disorder such as psoriasis, atopic dermatitis or non-melanoma skin cancer. The disclosure also provides methods of modulating gene expression through the application of intense, picosecond pulses of THz radiation to the skin of individuals afflicted with skin disorders such as non-melanoma skin cancers, psoriasis and atopic dermatitis.

Accordingly, the disclosure provides a means for modulating gene expression (e.g., in skin cells or in skin tissue) by providing a source of terahertz radiation, exposing skin cells or skin tissue to pulses of terahertz radiation in an amount sufficient to modulate gene expression where modulation of gene expression results in a reduction of a disease state in the skin cells or skin tissue.

In some embodiments, the terahertz radiation has a bandwidth of about 0.1 to about 10 terahertz, for example in some embodiments, about 0.2 to about 2.5 terahertz.

In some embodiments, the pulse of terahertz radiation is about 0.2 to about 100 picoseconds, about 5 to about 15 picoseconds, or about 10 picoseconds.

In some embodiments, the duration of exposure to terahertz radiation can be about 1 minute to about 30 minutes. In one specific embodiment, the duration of exposure to terahertz radiation can be about 10 minutes.

In some embodiments, the pulses of terahertz radiation have a pulse energy of about 0.01 to about 100 micro-joules, about 0.1 to about 2.0 micro-joules, or about 1.0 microjoule.

In some embodiments, the pulses of terahertz radiation have a repetition rate of about 1 hertz to about 100 megahertz. In one specific embodiment, the pulses of terahertz radiation have a repetition rate of about 1 kilohertz.

In some embodiments, the pulses of terahertz radiation are confined to a spot size of about 0.1 mm² to 100 mm².

In another embodiment, the pulses of terahertz radiation are raster scanned across a large area of the target (e.g., 100 mm² to two, three, four, or five times this area).

In some embodiments, the gene expression of the skin cells is down-regulated, or alternatively, up-regulated.

In some embodiments, the disease state is a skin disorder. In one specific embodiment, the disease state is a skin disorder such as psoriasis, atopic dermatitis, or other non-specific inflammatory skin conditions. In another embodiment the disease state is an inflammatory skin disease. In another embodiment the disease state is skin cancer. In some embodiments, the disease state is a non-melanoma skin cancer. In other embodiments, the skin cancer is basal cell carcinoma, squamous cell carcinoma, or other type of non-melanoma skin cancer. In another embodiment, the disease state is atopic dermatitis.

In various embodiments, therapeutic effect is achieved by modulating expression of specific disease-related target genes. In some embodiments, exposure to pulsed THz radiation causes down-regulation of genes that are up-regulated in diseased but not in healthy skin. In other embodiments, exposure to pulsed THz radiation causes up-regulation of genes that are down-regulated in diseased skin compared to healthy skin. In certain embodiments, at least one of the genes down-regulated is selected from the group consisting of S100P, S100A11, S100A12, S100A15, SPRR2A, SPRR3, SPRR1B, SPRR2B, SPRR2C, LCE3, LCE3D, CD24, DEFB103A, DEFB4, LOC728454, DEFB1, IVL, SERPINB3, SERPINB4 and SERPINB13. In other embodiments, at least one of the genes up-regulated is selected from the consisting of LCE1A, LCE1B, LCE1C, LCE1D, LCE1E, LCE1F and LCE5A.

In another embodiment, there is disclosed a method for treating skin cancer comprising the steps of a method for treating a patient with skin cancer using terahertz radiation, the method comprising the steps of providing a source of terahertz radiation, exposing a patient to pulses of terahertz radiation, wherein the pulses of terahertz radiation have a bandwidth of about 0.2-2.5 terahertz, a pulse energy of about 0.1-1.0 microjoules for about 30 minutes, the exposure to the terahertz radiation being in an effective amount to induce at least a 1.5 fold change in gene expression in the skin cancer cells as compared to a control sample not exposed to terahertz radiation; and where the change in gene expression results in a reduction in the level of skin cancer in the patient.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Intense THz pulses and skin tissue samples used in this study. (a) Electric field waveform of the THz pulse. (b) Spectral bandwidth of the THz pulse shown in A. (c) THz spot profile at the focus. The 1/e2 diameter of the THz beam is 1.5 mm. The 2 mm-diameter dashed circle shows the portion of the tissue that was excised for gene profiling. (d) Histology of an EpiDermFT tissue section (400× image, courtesy of MatTek). (e) Schematic illustration of the THz exposure procedure. The EpiDermFT tissue is in a single well insert placed at the focus of THz beam.

FIG. 2. Intense THz-pulse-induced gene expression in human skin. Venn diagrams summarizing differentially-expressed genes in EpiDermFT tissues exposed to either 1.0 μJ or 0.1 μJ THz or UVA (400 nm, 0.024 μJ) pulses, as described in Materials and Methods. Genes with a False Discovery Rate (FDR)-adjusted p-value <0.05 and log 2 fold change >0.6 (1.5× change) were considered differentially expressed. Left diagram: down-regulated genes. Right diagram: up-regulated genes. The complete lists of the affected genes, the fold change and p-values are given in FIG. 4, FIG. 5 and FIG. 6.

FIG. 3. Differentially expressed Epidermal Differentiation Complex (EDC) genes and selected other genes associated with non-melanoma skin cancer or inflammatory skin diseases. (a) EDC genes that were either up-regulated (green rectangles) or down-regulated (red rectangle) after exposure of skin tissues to either 1.0 μJ or 0.1 μJ THz pulse energies, or UVA pulses. Unaffected genes are depicted as white rectangles. (b) log 2 fold changes for differentially-expressed genes belonging to four families of EDC genes (FLG-like, LCE, 5100, and SPRR). (c) Other genes (selected) involved in dermatological diseases and cancer, whose expression levels were altered by exposure to both THz pulse energy regimes. In (b) and (c), error bars indicate corresponding standard deviations, and in some cases are smaller than the symbol size. Mean values, log 2 fold changes relative to control mean values, and FDR-adjusted p-values for genes in (b) and (c) are given in FIG. 7. Individual replicates, mean values and corresponding standard deviations are given in FIG. 8.

FIG. 4. Genes differentially expressed following exposure to 1.0 μJ THz pulses.

FIG. 5. Genes differentially expressed following exposure to 0.1 μJ THz pulses.

FIG. 6. Genes differentially expressed following exposure to UVA (0.024 μJ 400 nm) pulses.

FIG. 7. Differentially expressed EDC genes and selected other genes associated with non-melanoma skin cancer or inflammatory skin diseases, as shown in FIGS. 3b and 3c.

FIG. 8. RNA expression measured for differentially expressed EDC genes and selected other genes associated with non-melanoma skin cancer or inflammatory skin diseases. (a) Replicates, mean values and standard deviations for control and 1.0 μJ THz pulse-exposed tissues. Symbols of up-regulated genes are highlighted in red, down-regulated genes in green, and unaffected genes are shown in black. (b) Replicates, mean values and standard deviations for control and 0.1 μJ THz pulse-exposed tissues. Symbols of up-regulated genes are highlighted in red, down-regulated genes in green, and unaffected genes are shown in black. (c) Replicates, mean values and standard deviations for control and UVA-exposed tissues. Symbols of up-regulated genes are indicated as red (“[R]” after the Gene symbol), down-regulated genes are indicated as green (“[G]” after the Gene symbol), and unaffected genes are shown in black (all others not marked with [R] or [G] after the Gene symbol).

### DETAILED DESCRIPTION

Non-melanoma skin cancer (NMSC) and psoriasis are skin disorders that affect a large portion of the population globally. NMSC affects about 3.5 million individuals in the United States alone, while psoriasis affects between 1-3% of the population of the United States. Current methods for treating these skin disorders include topical applications, systemic application, radiation therapy, UV radiation or skin removal. Unfortunately, each of these methods has disadvantages such as pronounced side effects or scaring. Therefore, there is a need for a method to overcome these difficulties.

Broadband terahertz (THz) radiation resides between the infrared and microwave wavelengths (0.1-10 THz) of the electromagnetic spectrum. THz photons are low energy, with photon energies typically between 0.5 and 15 meV, and therefore represent radiation. This is in contrast to ionizing radiation such as Ultra-violet radiation, having high energy photons around 3.0 eV. Ionizing radiation exerts detrimental effects on living cells by breaking chemical bonds in DNA and other biological molecules. The non-ionizing nature of THz radiation makes it a useful tool in clinical applications. Accordingly, ultrafast lasers emitting THz radiation have been used as non-invasive tools for cancer diagnosis, intra-operative tumor margin identification, burn assessment, and in vivo skin and cornea hydration sensing.

Although THz radiation can have thermal effect on biological tissue due to absorption of the radiation by water, such effects are typically limited to instances of continuous wave excitation. More recent theoretical modeling and other experiments suggest many important biomolecules (e.g., nucleic acid, proteins) have intrinsic vibrational resonances in the THz range. It is thought that resonant coupling of THz radiation with the intrinsic vibrational modes of biomolecules may affect the conformational state of the biomolecule, thereby impacting cellular function. Moreover, THz radiation only penetrates the human body to a depth of a few millimeters, thereby avoiding any irradiation of vital organs. These characteristics make THz radiation ideal for the treatment of skin disorders.

Described herein is a method for treating skin disorders including, but is not limited to the steps of: 1) providing a source of terahertz radiation, 2) exposing skin cells to pulses of THz radiation and 3) modulating the gene expression to cause a reduction of a disease state, or prevent an increase in the disease state in the skin cells.

In a more specific embodiment, there is provided a method for treating skin cancers that includes the steps of exposing a patient to a source of terahertz radiation wherein the pulses of terahertz radiation have a bandwidth of about 0.2-2.5 terahertz, a pulse energy of about 0.1-1.0 microjoules and an exposure time of about 30 minutes. The exposure to the terahertz radiation is sufficient to induce at least a 1.5 fold change in gene expression in the skin cancer cells as compared to a control sample not exposed to terahertz radiation, resulting in a reduction in the level of skin cancer in the patient.

THz radiation can be generated by any method known to one skilled in the art such as but not limited to Far Infrared Lasers, Quantum Cascade Lasers and Free electron Lasers. Furthermore, terahertz radiation can be generated using a Ti:Saphire femtosecond lasers that are directed into an electro-optic crystal such as a zinc blend crystal (e.g., zinc telluride crystal, gallium arsenide crystal) or lithium crystals such as lithium niobate. In another approach, a photoconductive dipole antenna can be used. In a further embodiment, terahertz radiation can be obtained by mixing multiple emissions from near-infrared diode lasers. Other sources of THZ radiation include backward wave oscillators, gyrotrons, Gunn diodes, frequency multiplier units based on Schottky Barrier diodes, impact ionization avalanche transit-time devices (IMPATTs), tunneling transit time diodes (TUNNETT), and resonant tunneling diodes. Preferably, the source of THz radiation is hand held or capable of being set up on a bench top. Generally, the source of THz radiation is held or positioned in close proximity to the sample to be treated. In some embodiments, the source of THz radiation is positioned so that the radiation beam covers a spot size of about 1.5 mm to about 2 mm. In other embodiments, larger or smaller areas can be subject to the THz radiation with spot diameters, for example of 0.1, 5, 10, 100, 1000, 10000 fold the diameter of the above referenced spot size (1.5-2 mm).

In some embodiments, the skin cells, tissue or patient are subjected to pulses of THz radiation. The duration of THz pulses can be about 1-15 picoseconds, preferably about 1-10 picoseconds, more preferably about 1-5 picoseconds and even more preferably about 1 picosecond. Moreover, in some embodiments, the range of THz radiation can range between 0.1-10 THz, preferably about 0.1-5 THz and more preferably about 0.2-2.5 THz. In other embodiments, the repetition rate for THz pulses is generally about 1-100000 kilohertz, about 1-50000 kilohertz, about 1-10000 kilohertz, about 1-1000 kilohertz, about 1-500 kilohertz, about 1-100 kilohertz, about 1-50 kilohertz, or about 1 kilohertz.

In some embodiments, pulse energies may range between 0.1-100 microjoules, and preferably about 0.1-10 microjoules, and more preferably about 0.1-2 microjoule, and even more preferably about 0.1-1.0 microjoules. In further embodiments, the THz radiation can have a peak electric field of about 220 kV/cm for a 1.0 microjoule pulse energy and about 70 kV/cm for a 0.1 microjoule pulse energy.

In some embodiments, the total exposure to THz radiation pulses can be about, 1-120 minutes, or about 1-60 minutes, about 1-30 minutes and preferably about 2-10 minutes.

In still further embodiments, the energy density per pulse of THz radiation can be about 6 microjoules/cm² for a 0.1 microjoule THz pulse and about 60 microjoules/cm² for a 1 microjoule THz pulse.

The application of THz radiation to the skin cells can modulate gene expression (up-regulation or down-regulation) in the area of exposure. Although the mechanism for this action is not entirely known, THz radiation is thought to cause conformational changes in the cellular machinery. For example, THz pulses can cause local melting (AKA breathing) of double-stranded nucleic acid, thereby facilitating gene transcription. THz radiation can also cause the activation or repression of transcription factors by inducing a conformation change in various proteins, thereby allowing or disrupting interaction with other biomolecules.

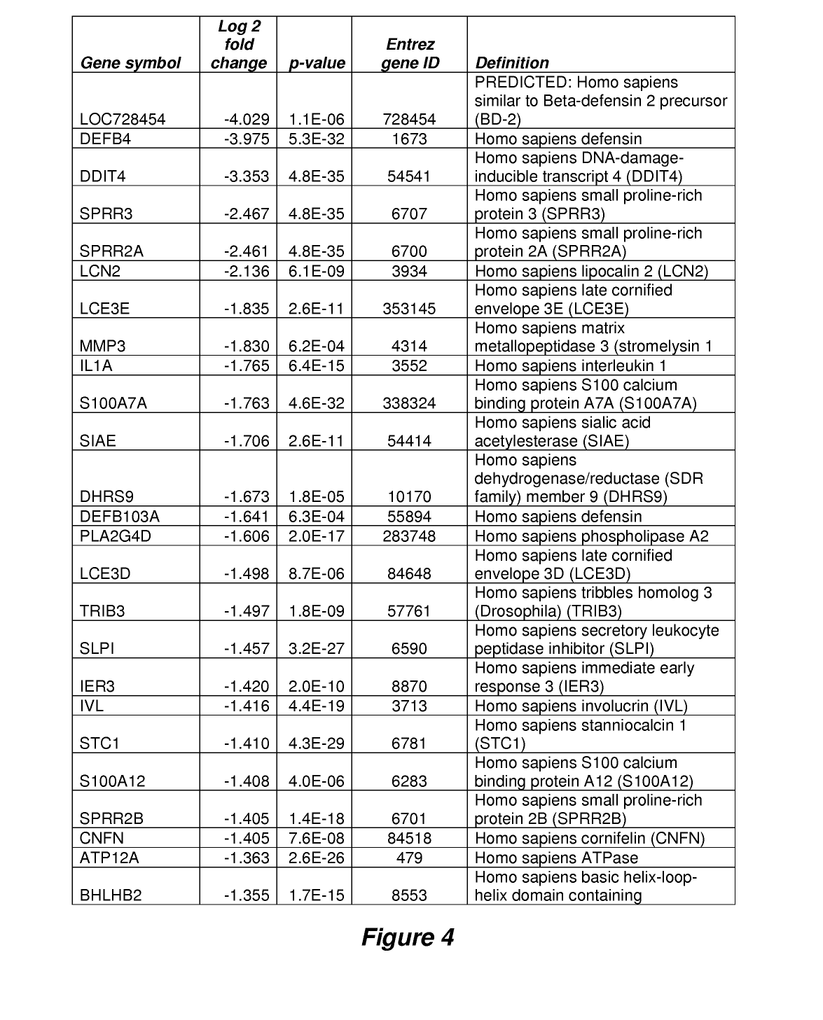

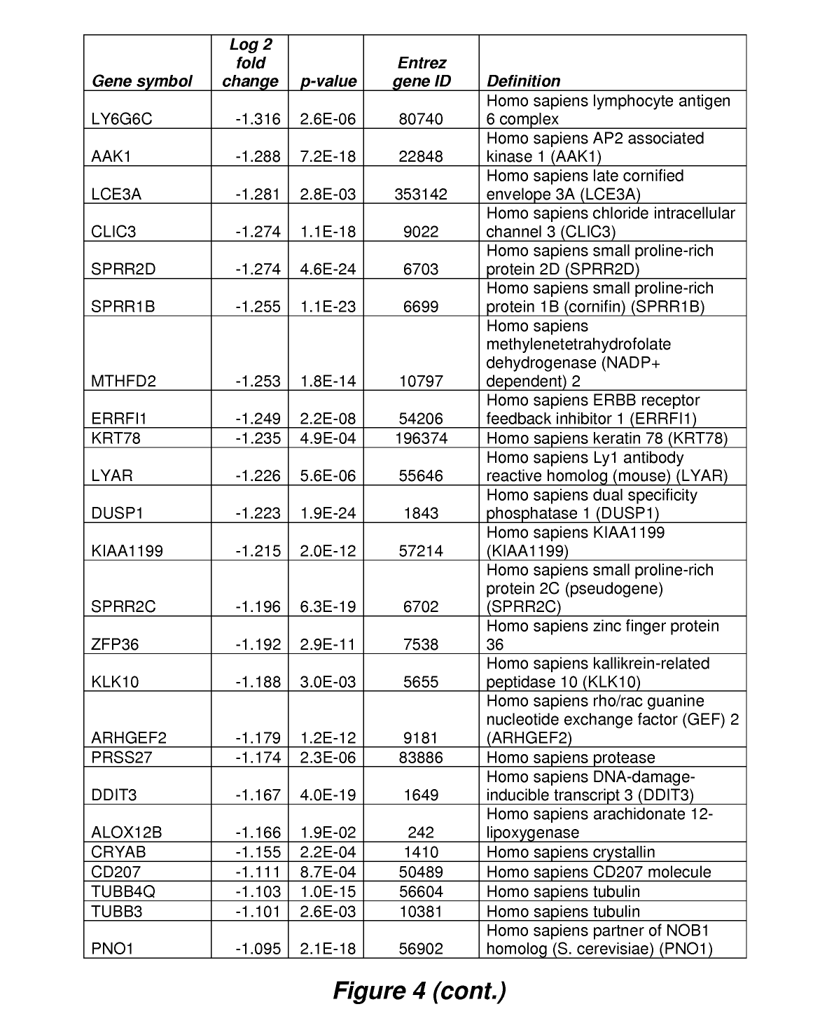

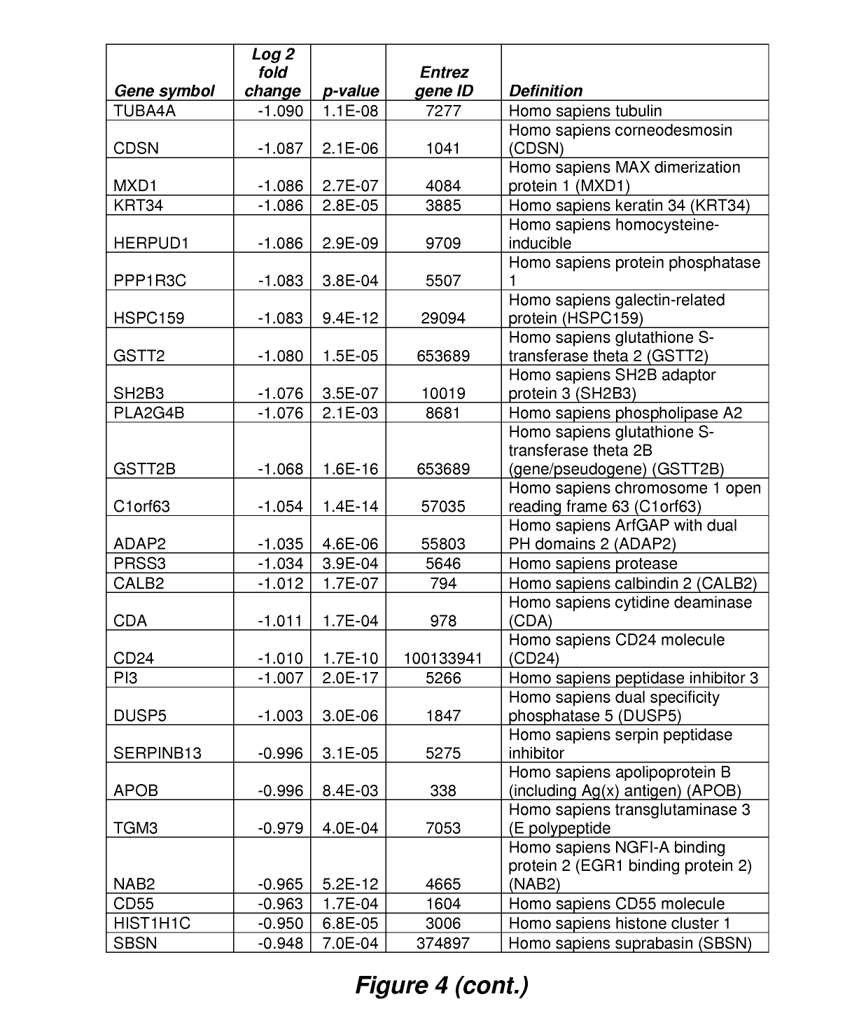

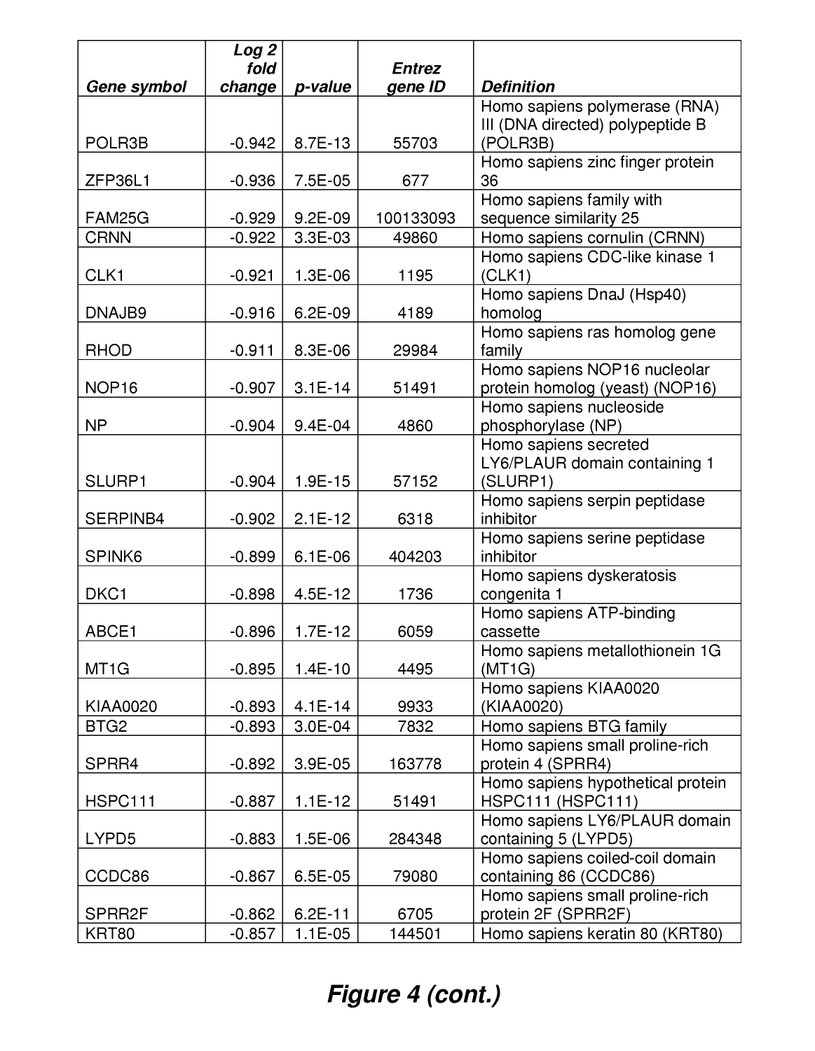

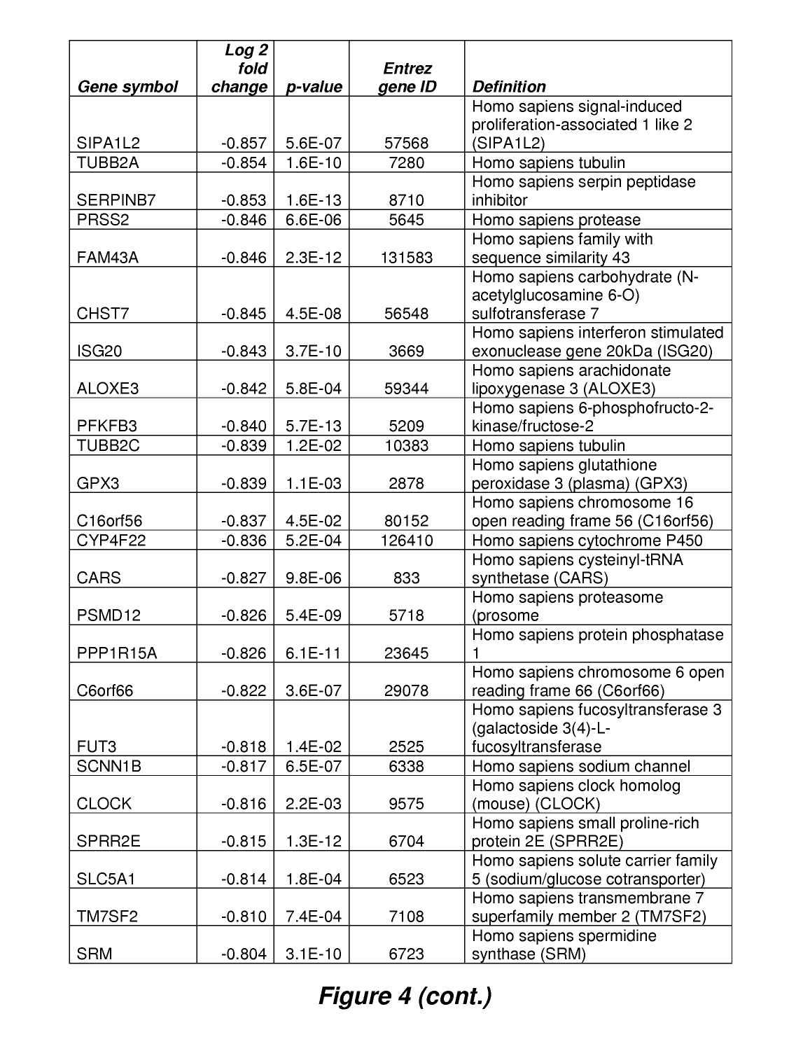

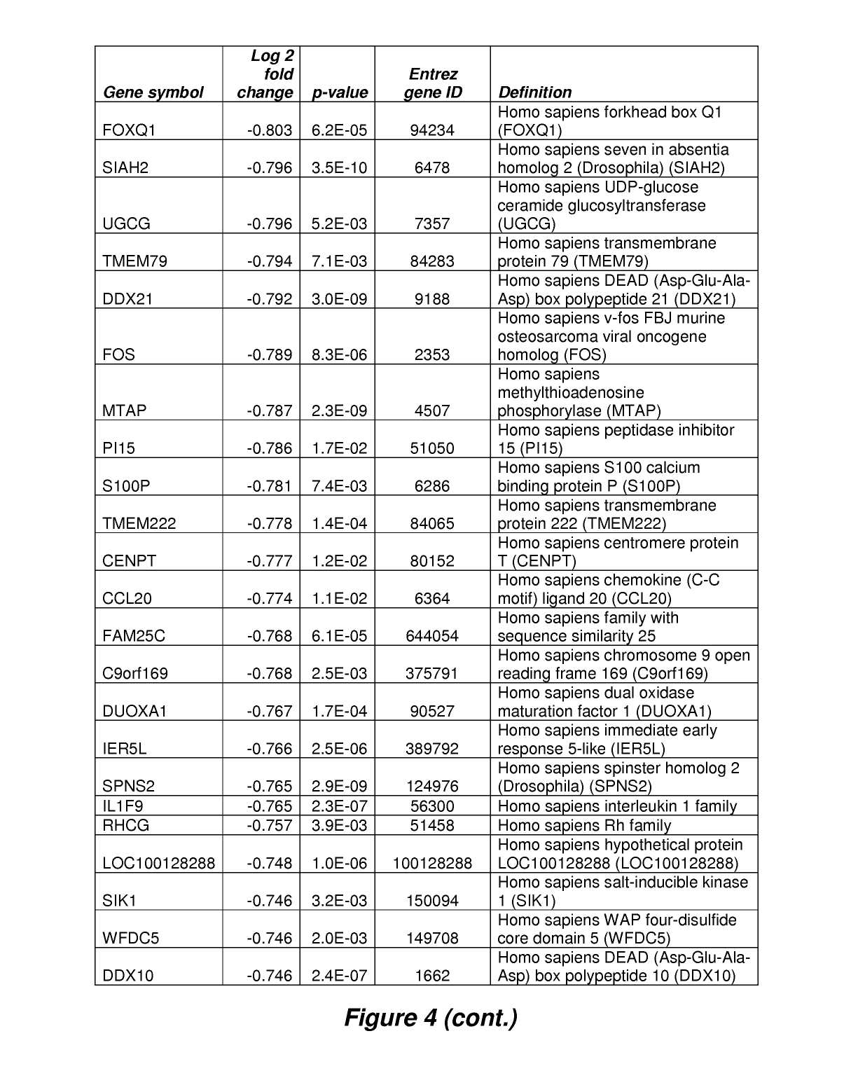

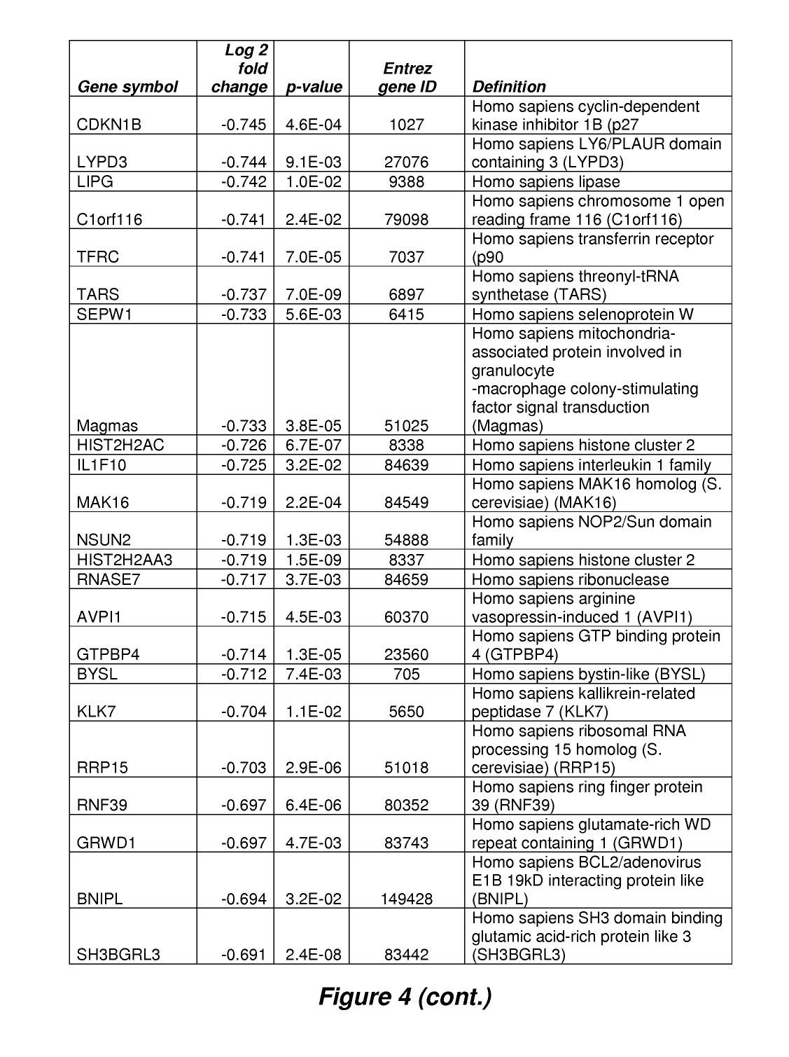

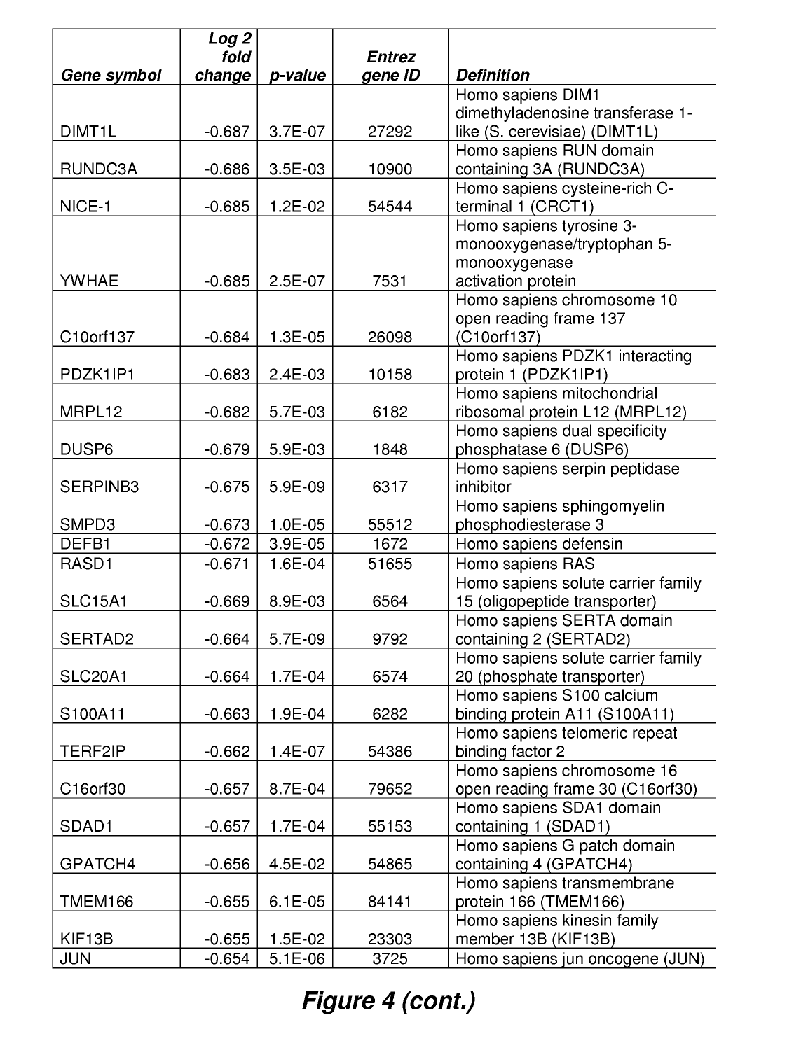

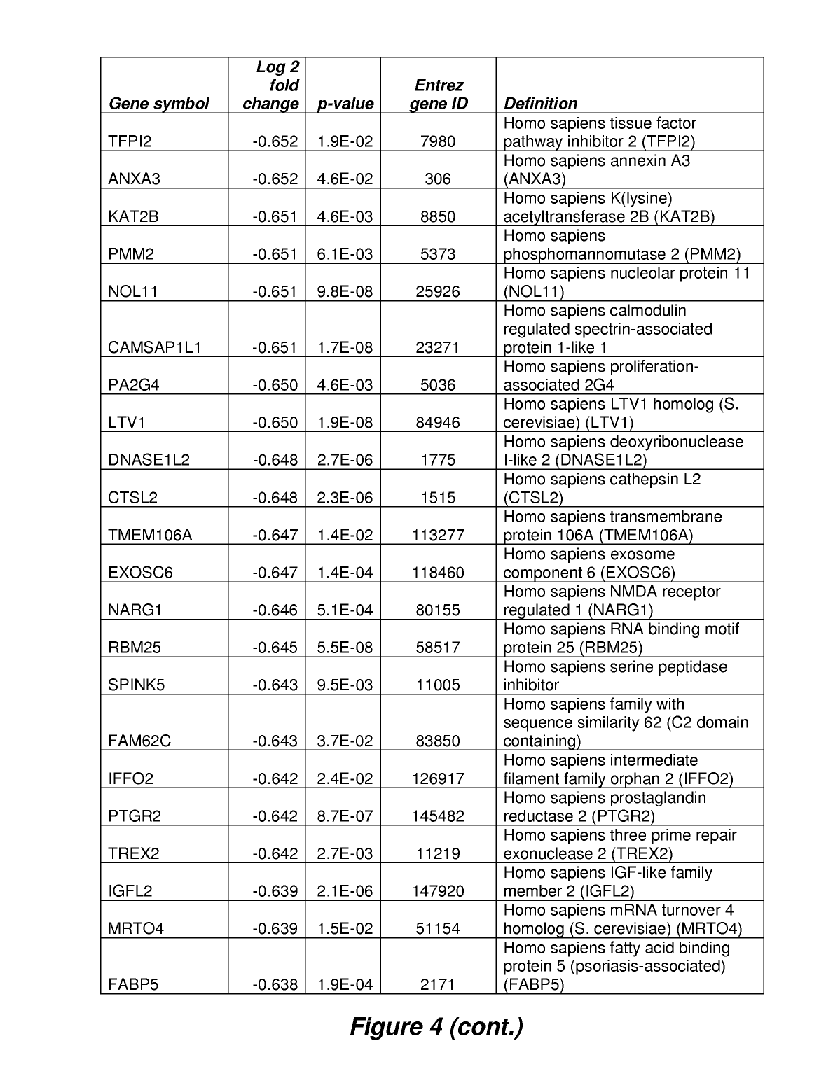

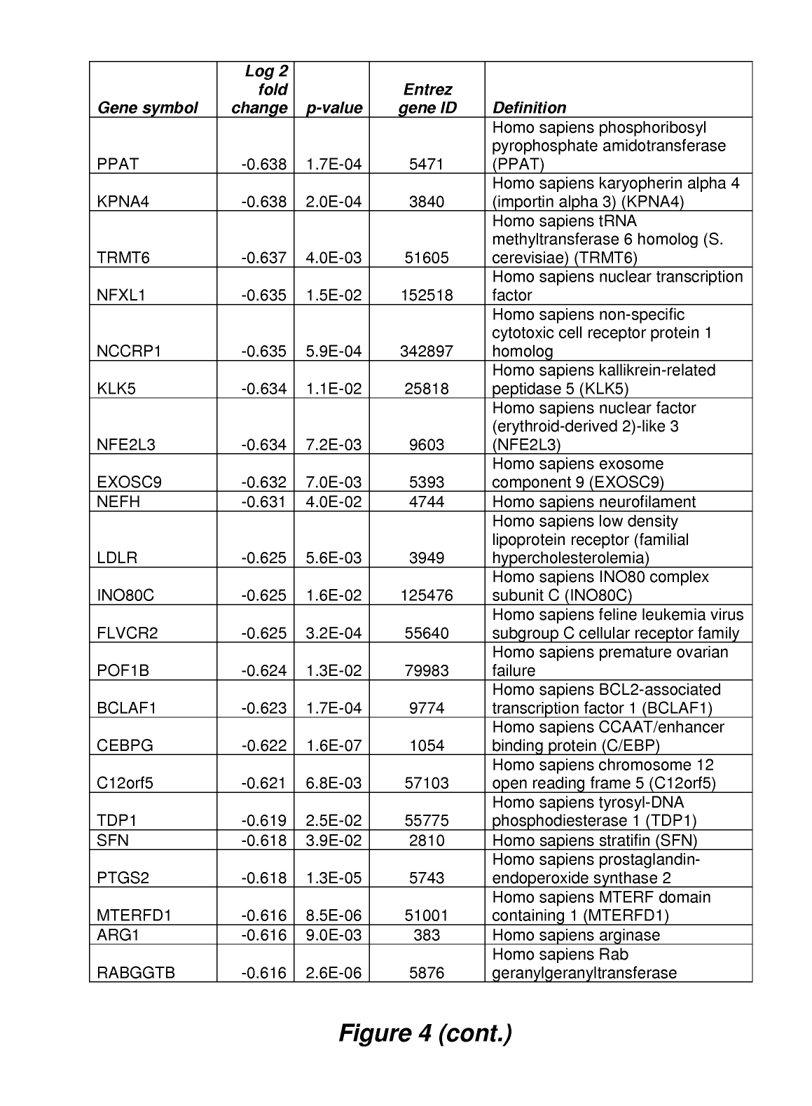

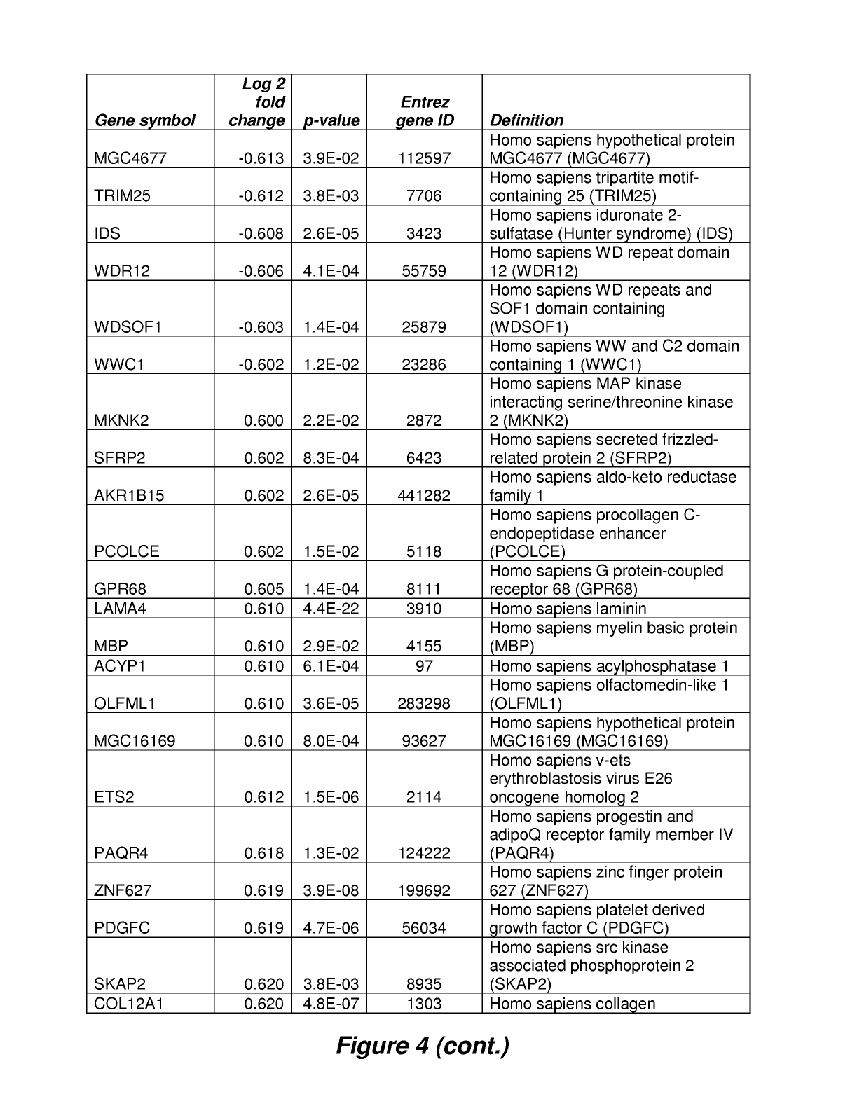

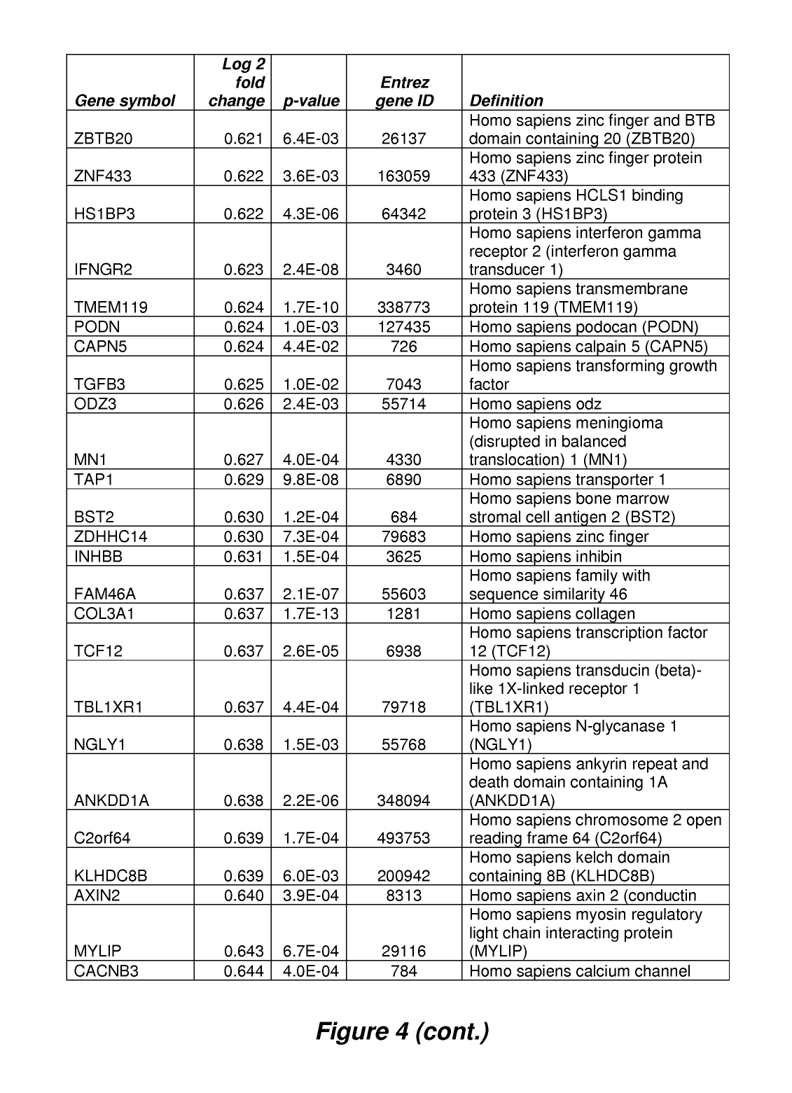

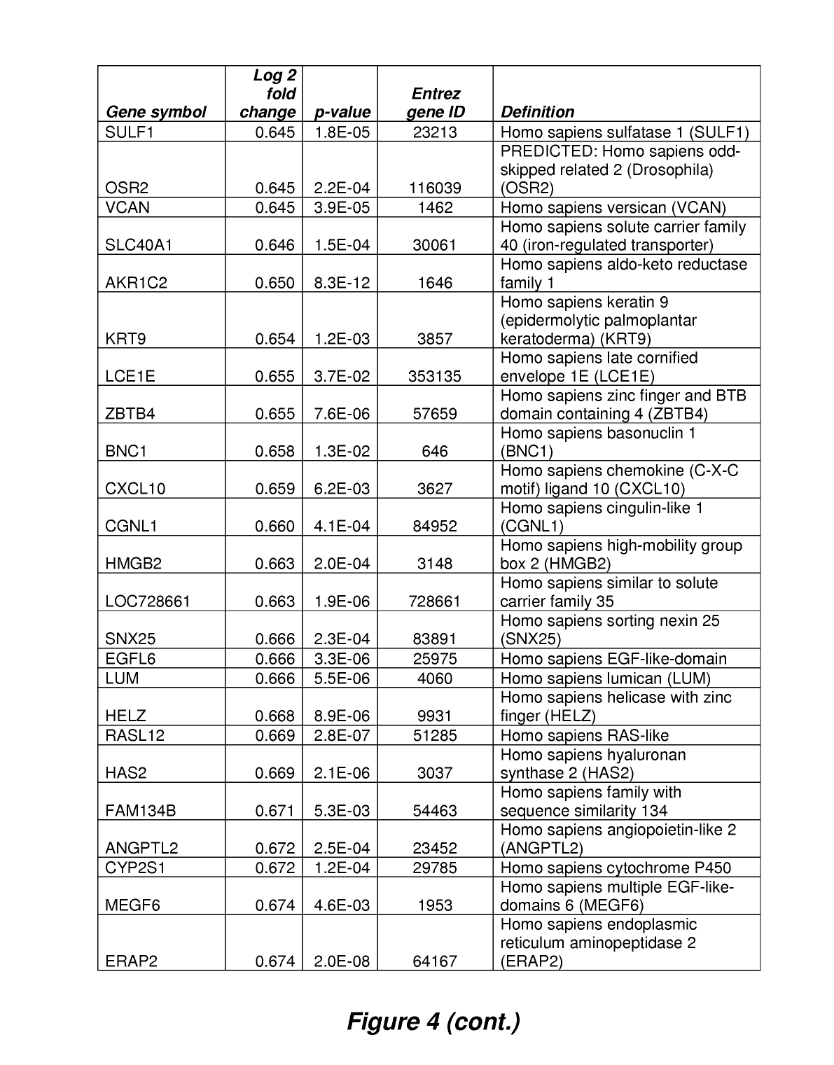

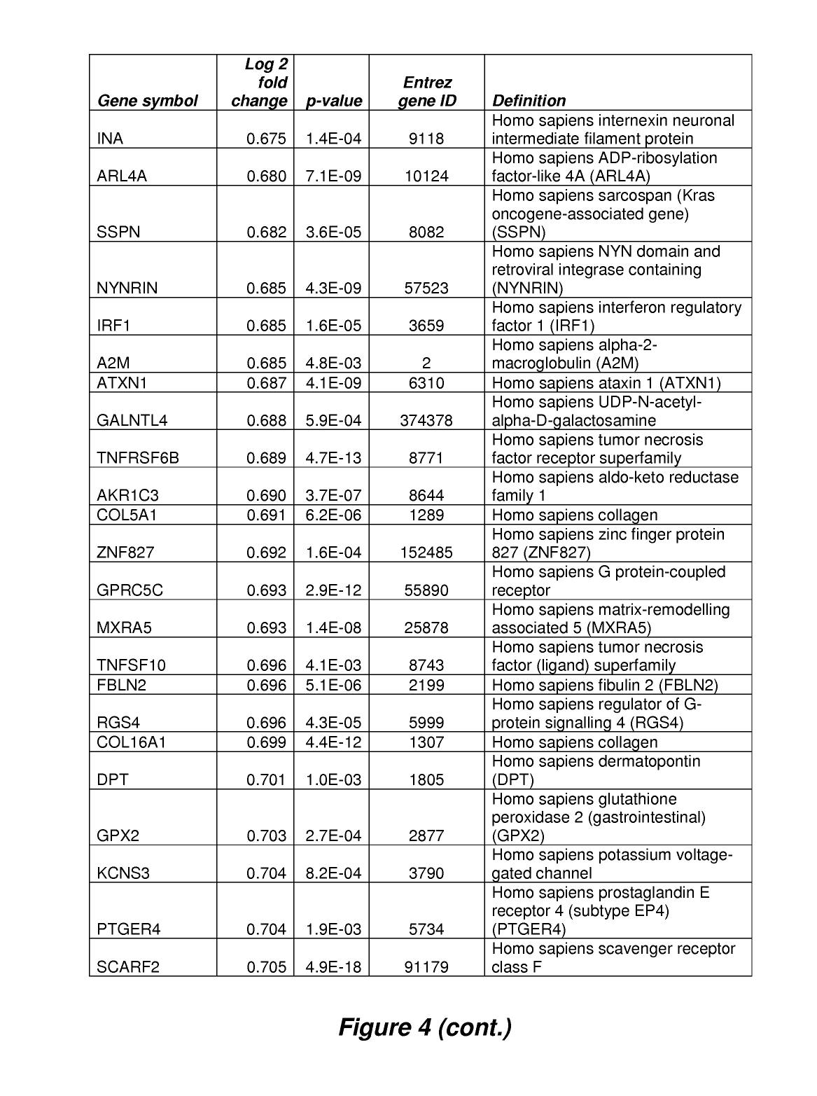

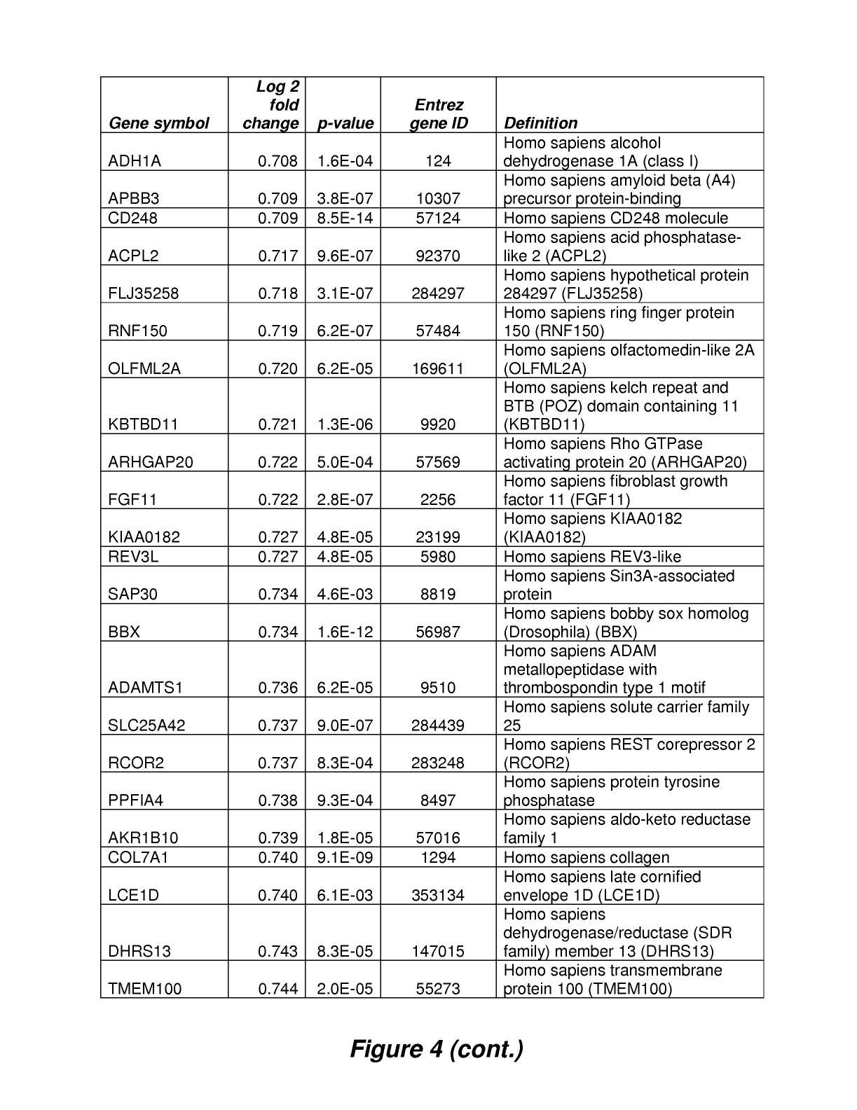

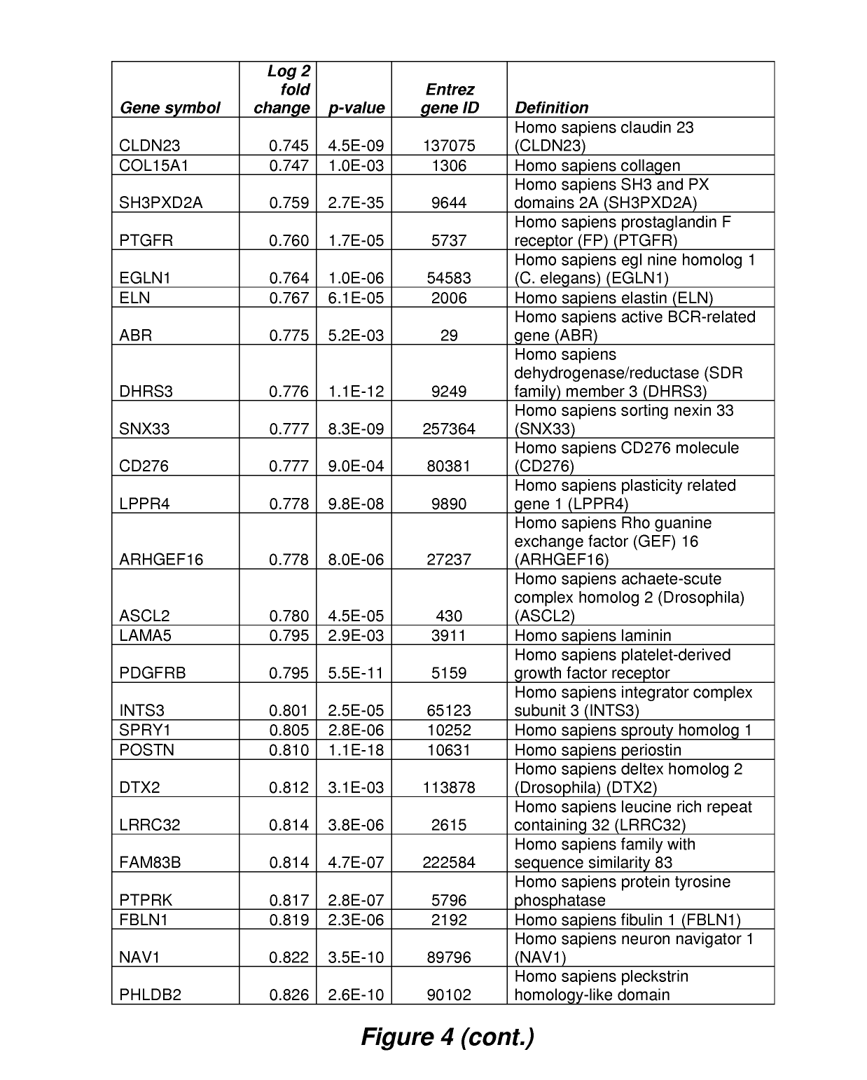

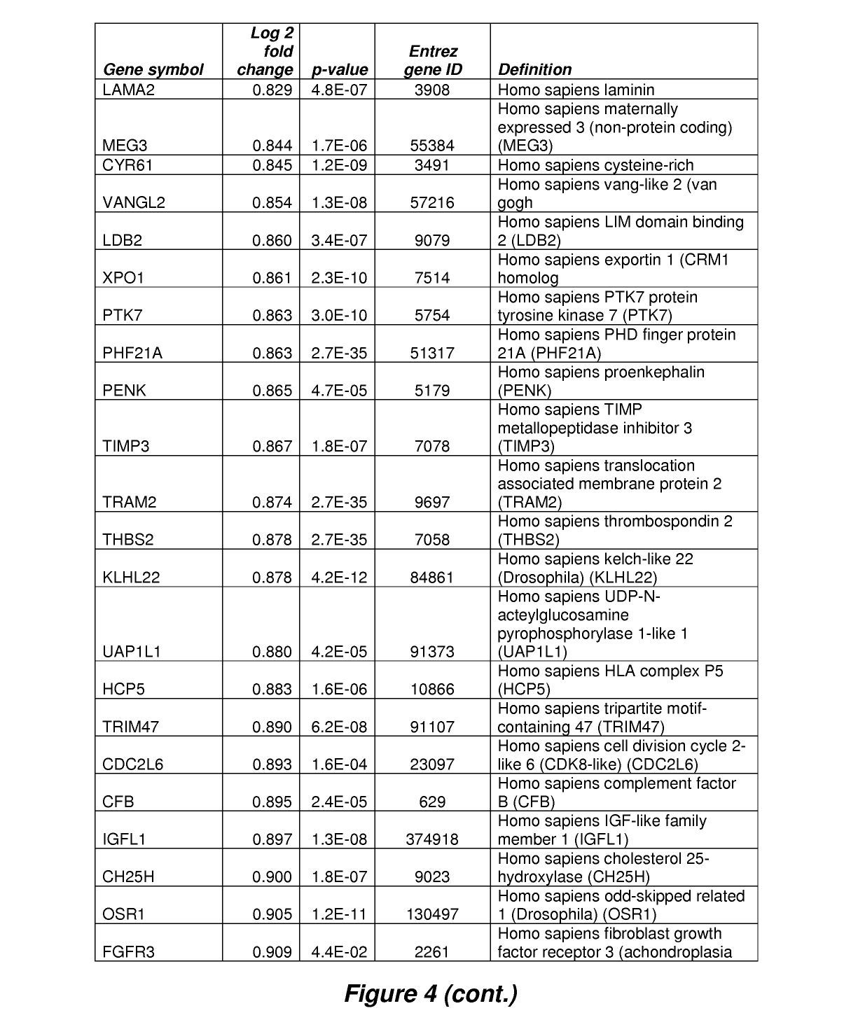

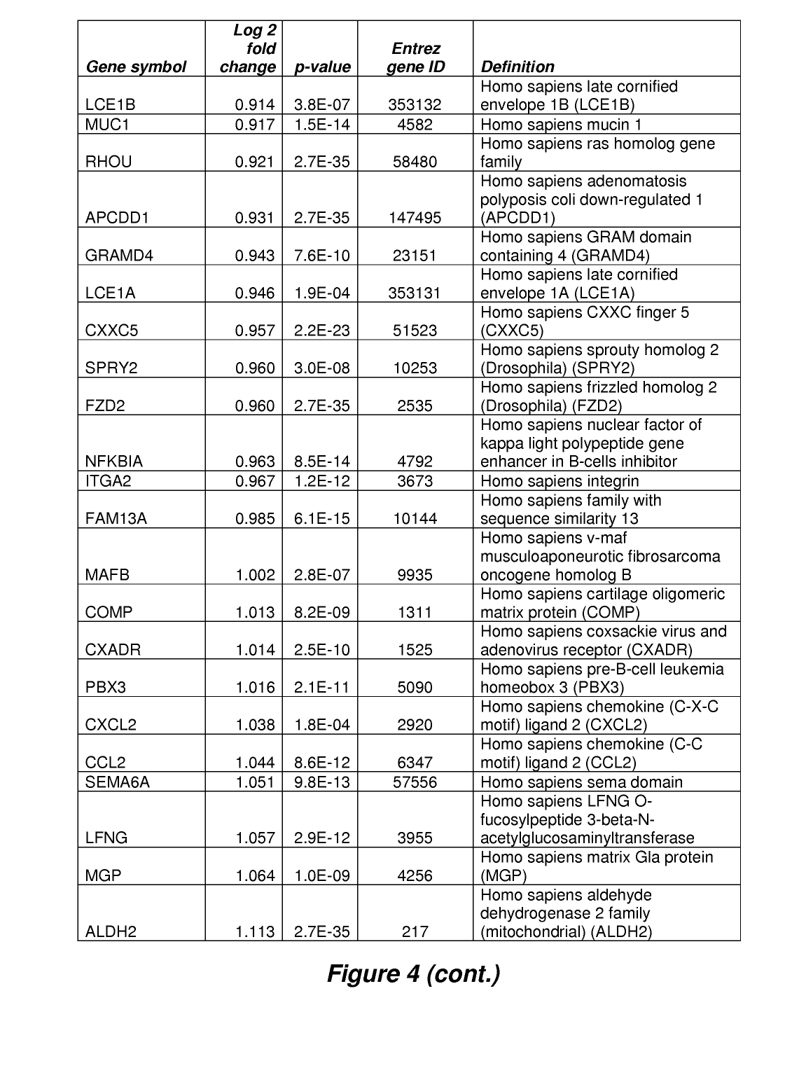

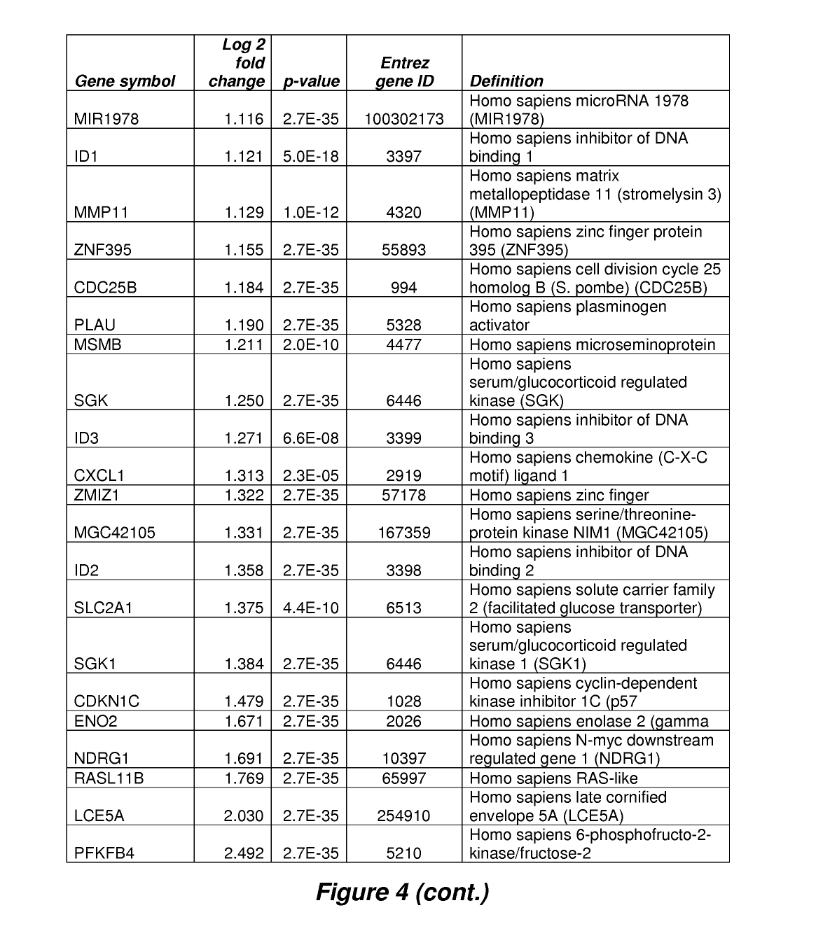

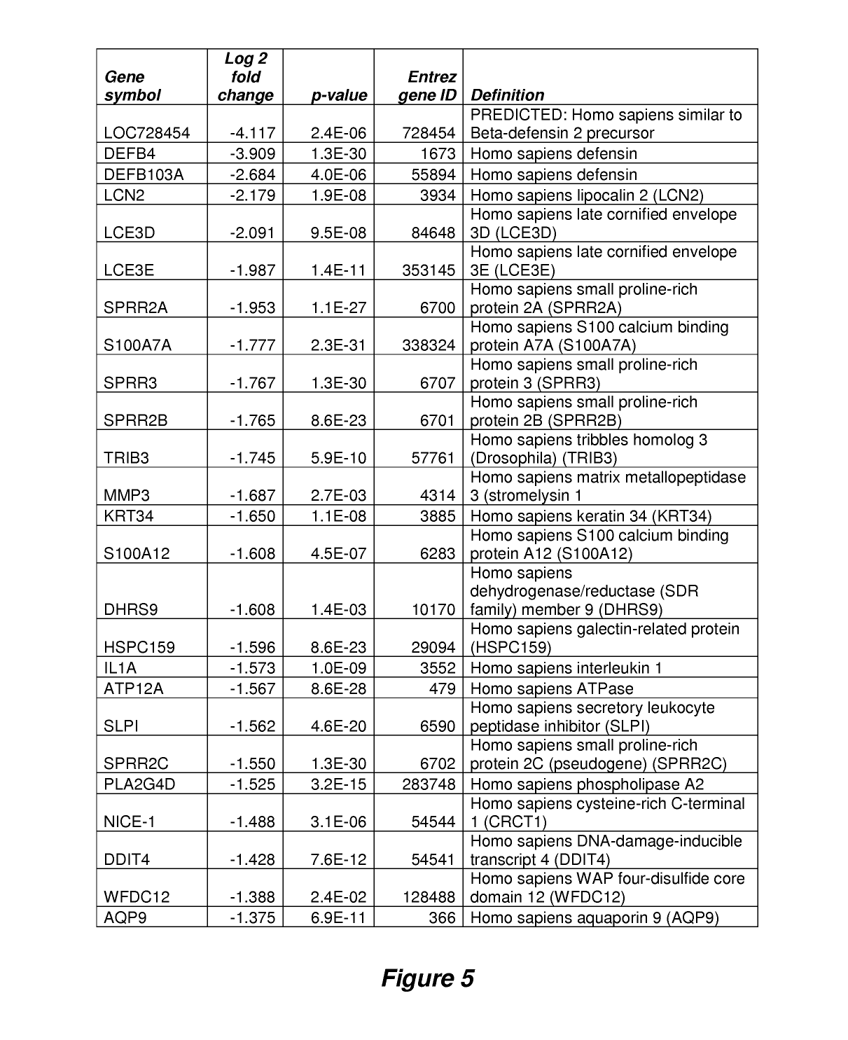

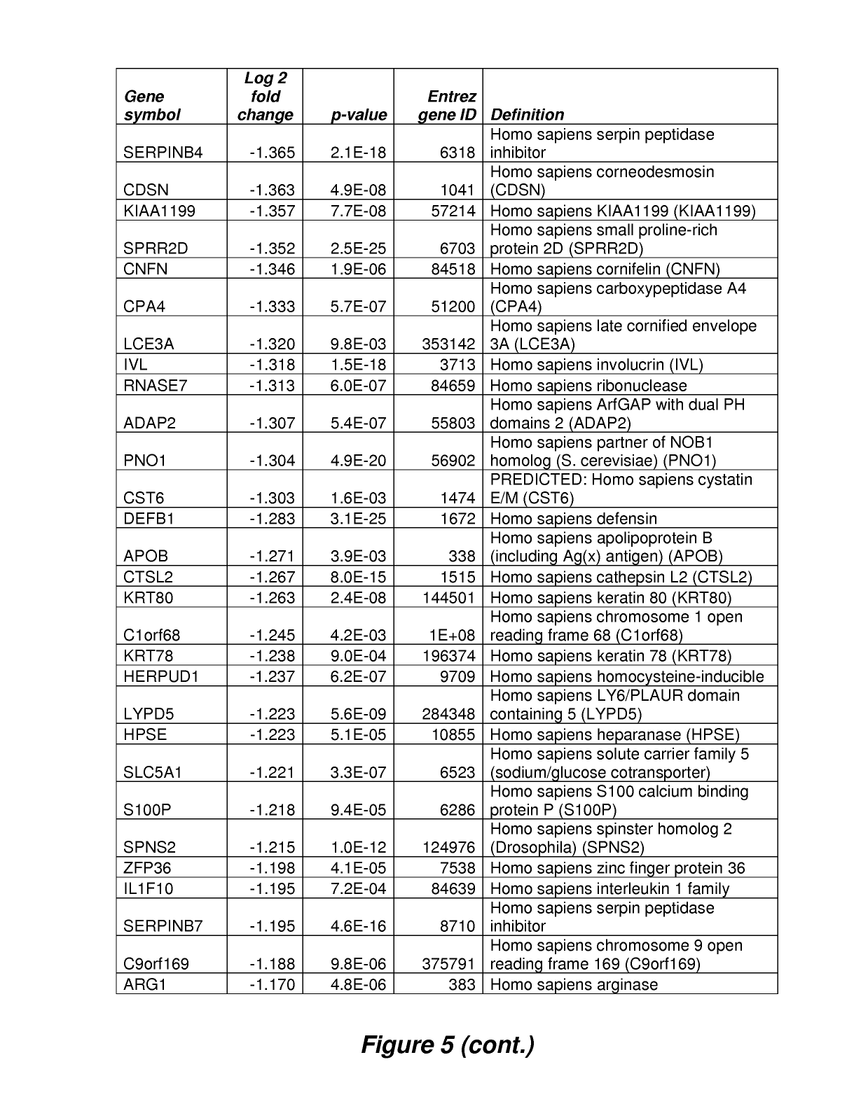

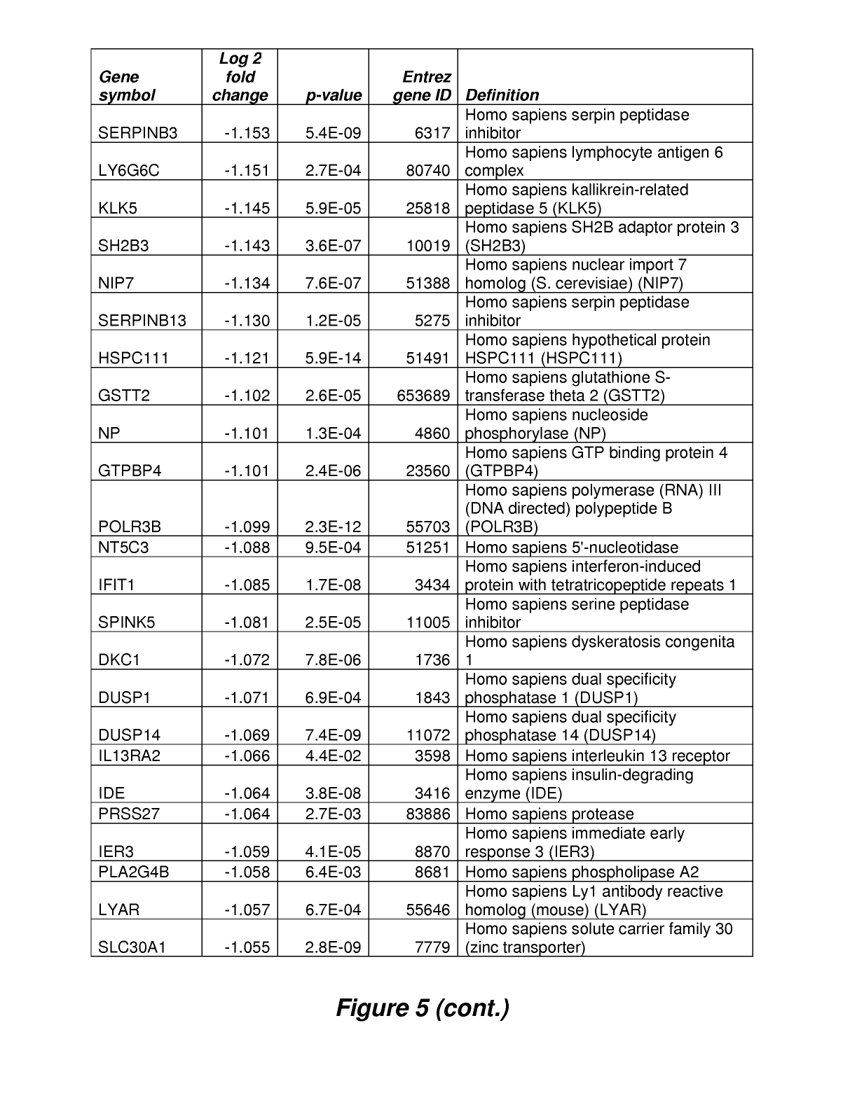

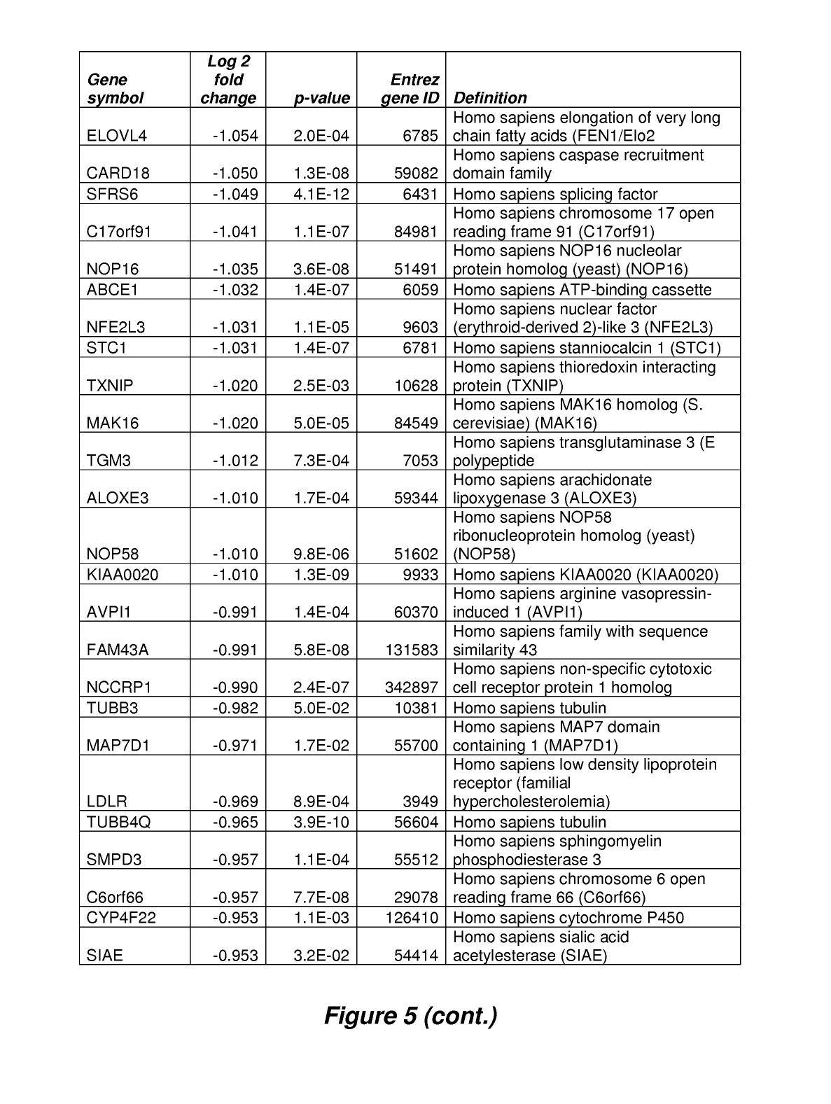

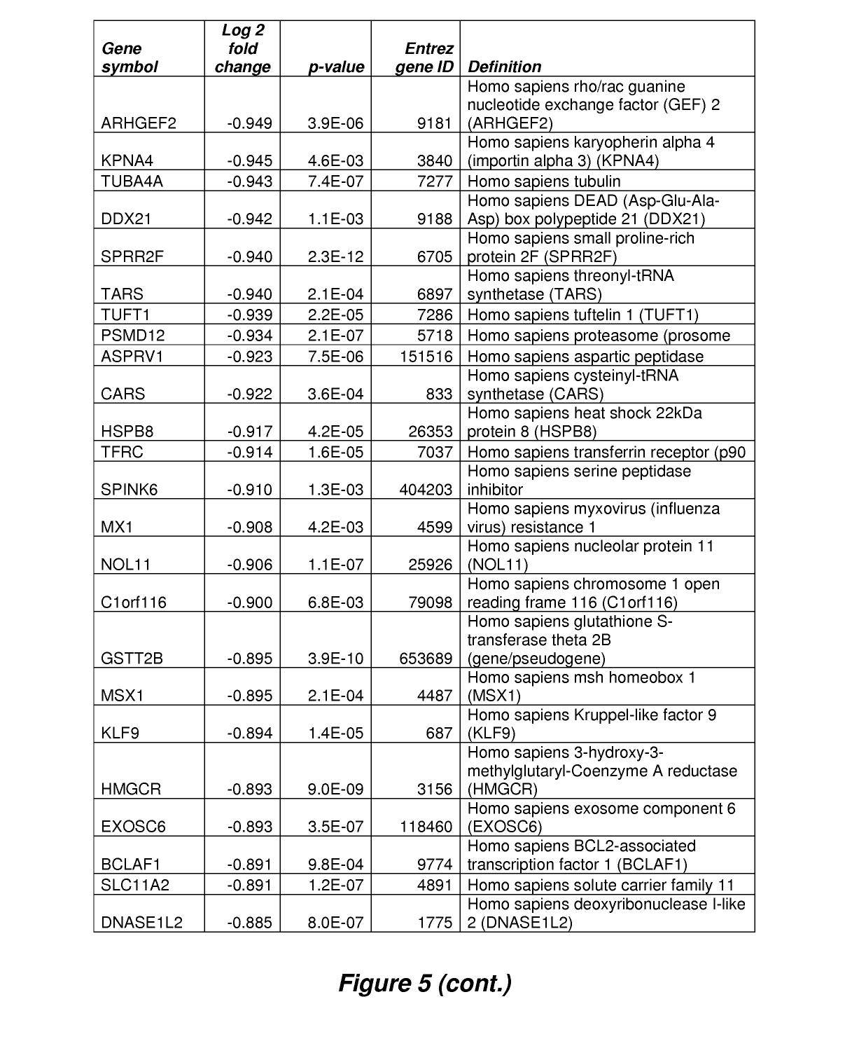

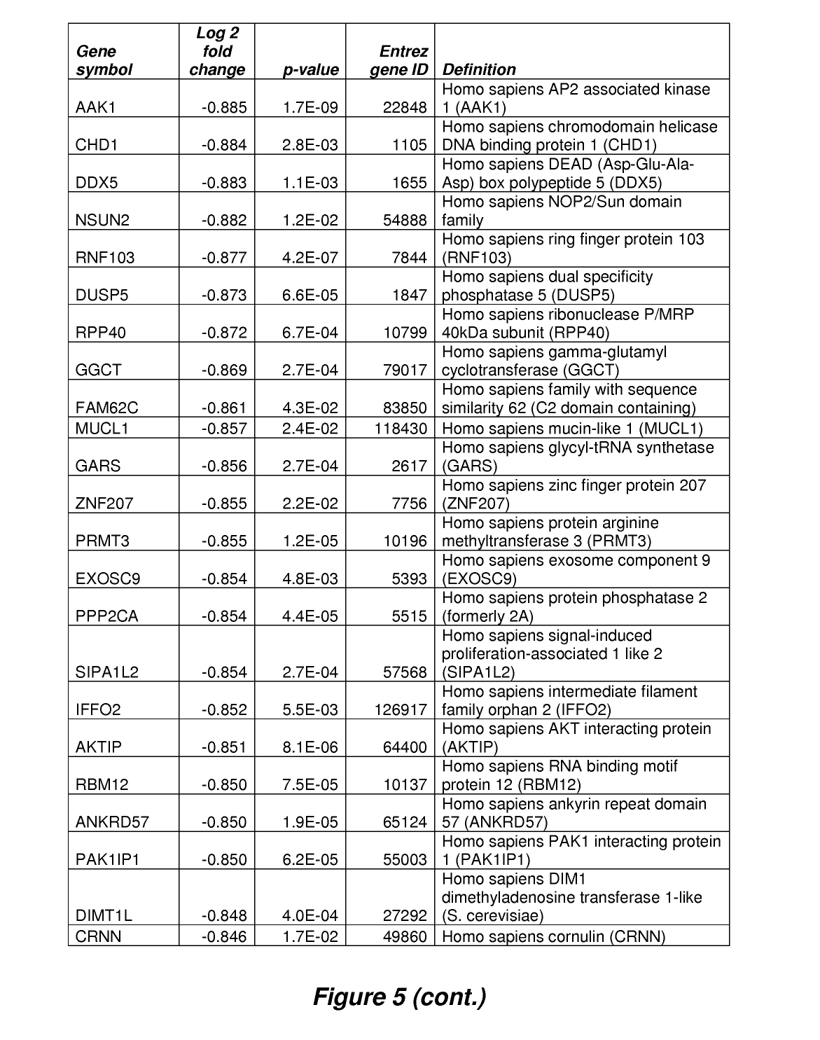

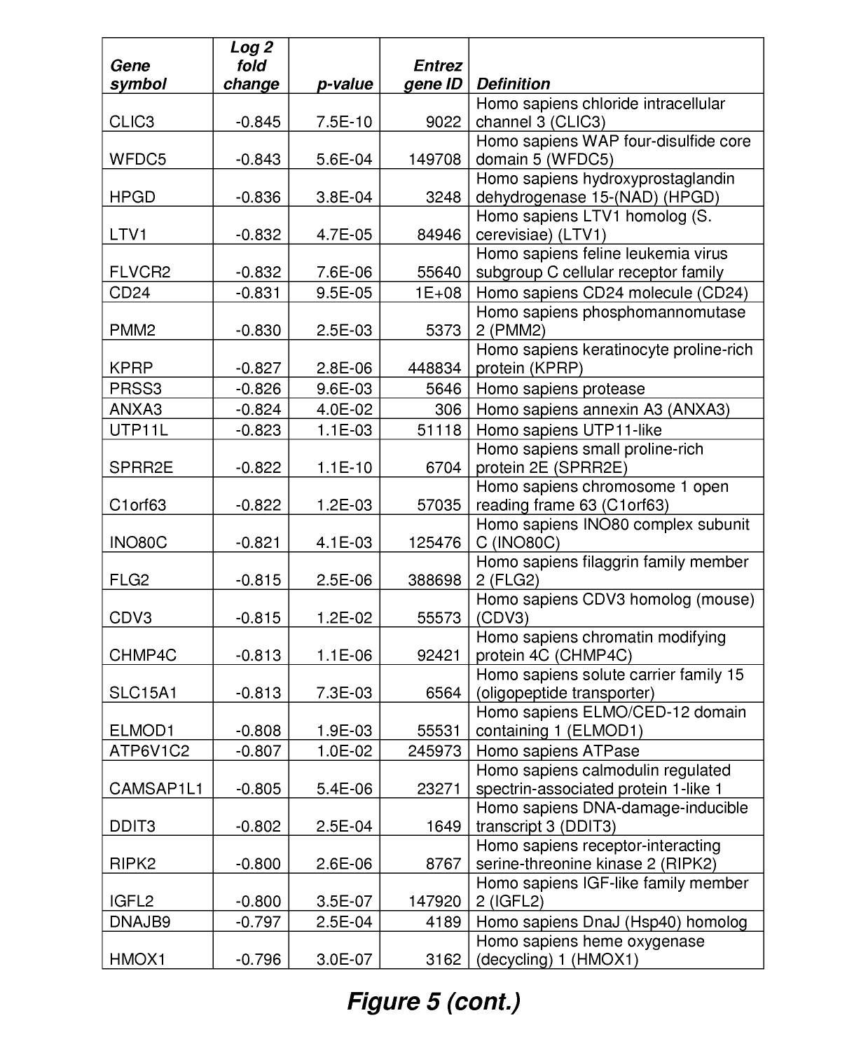

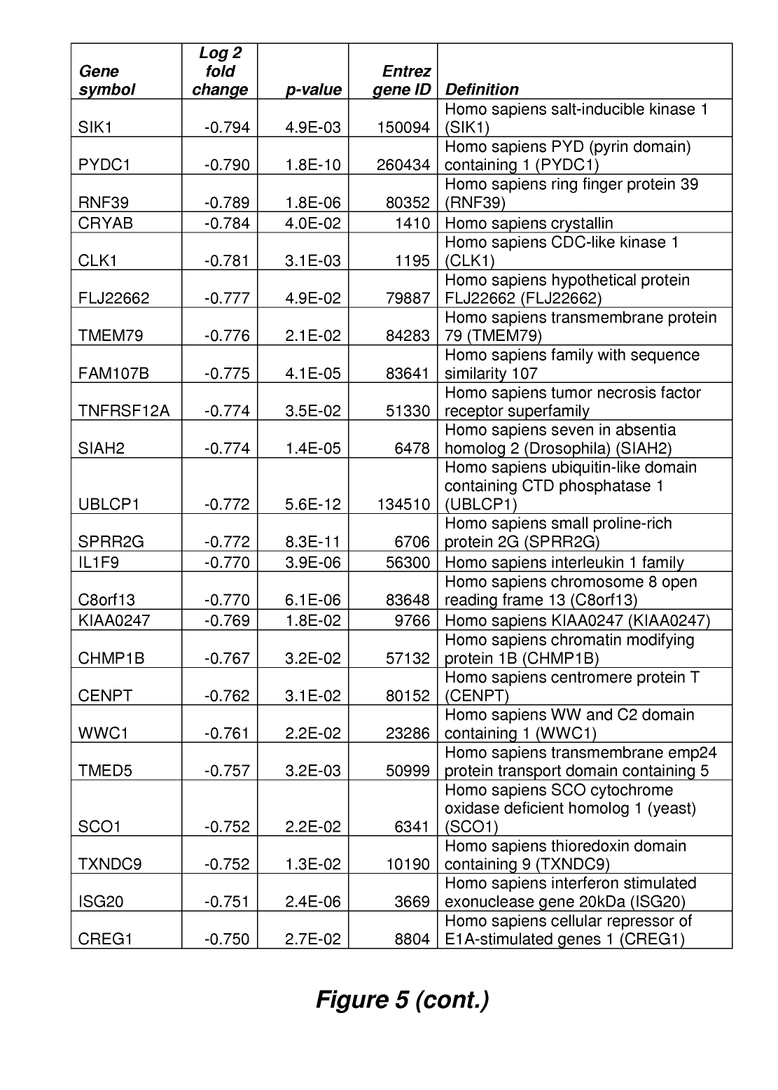

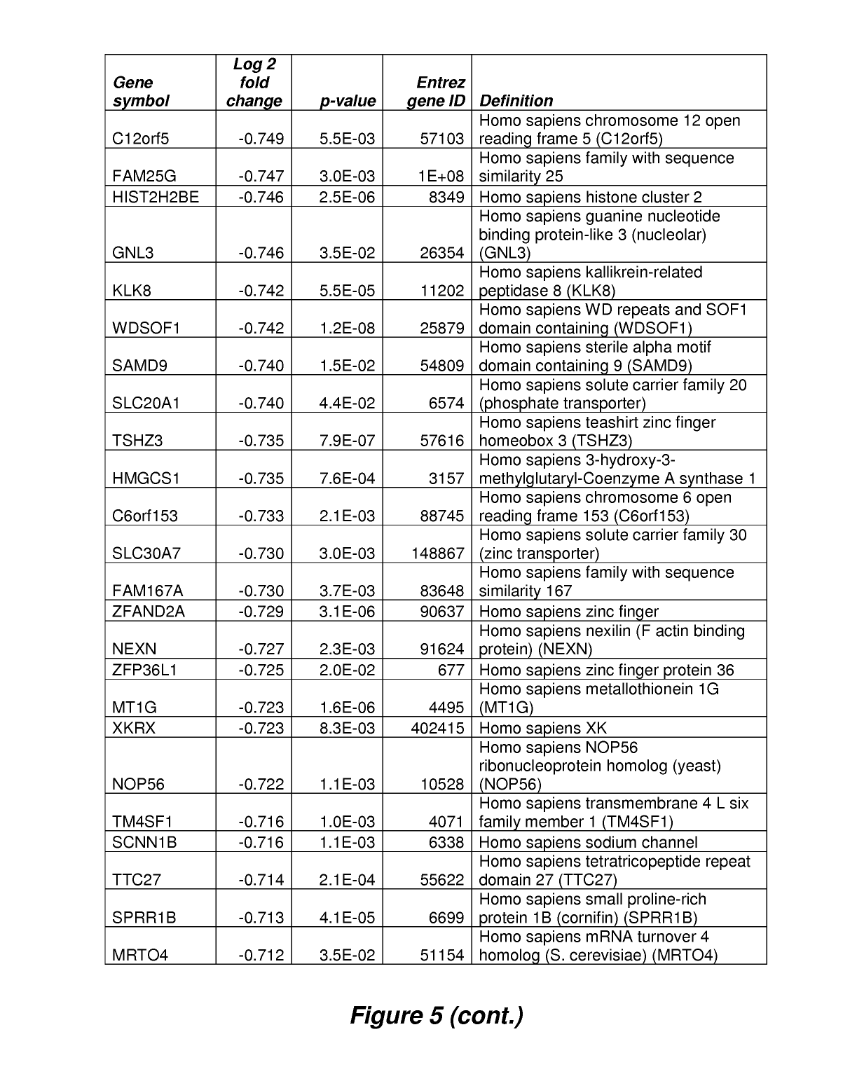

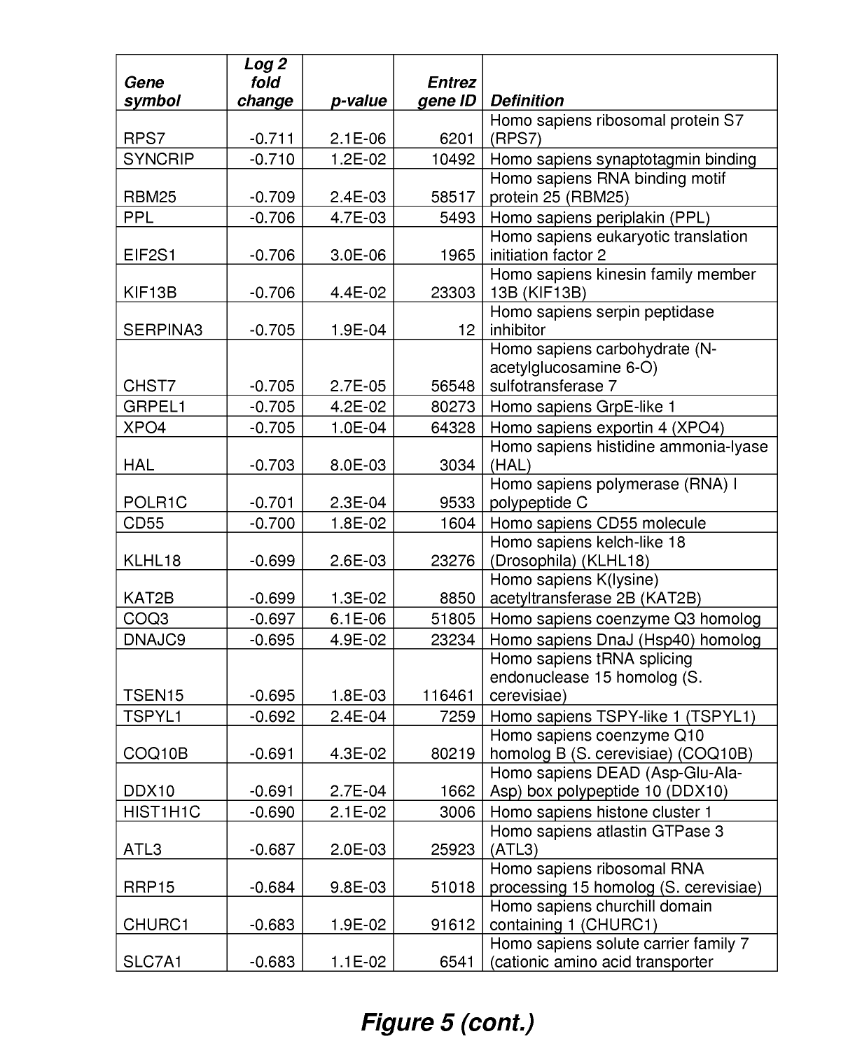

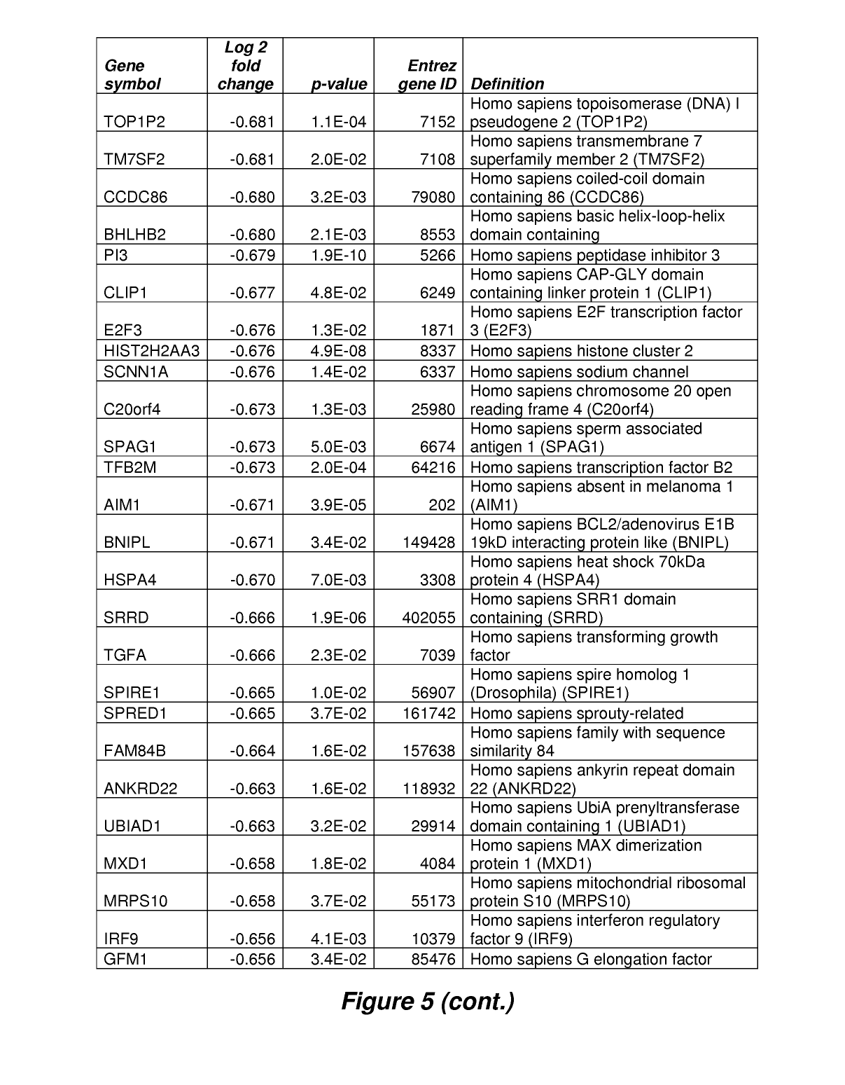

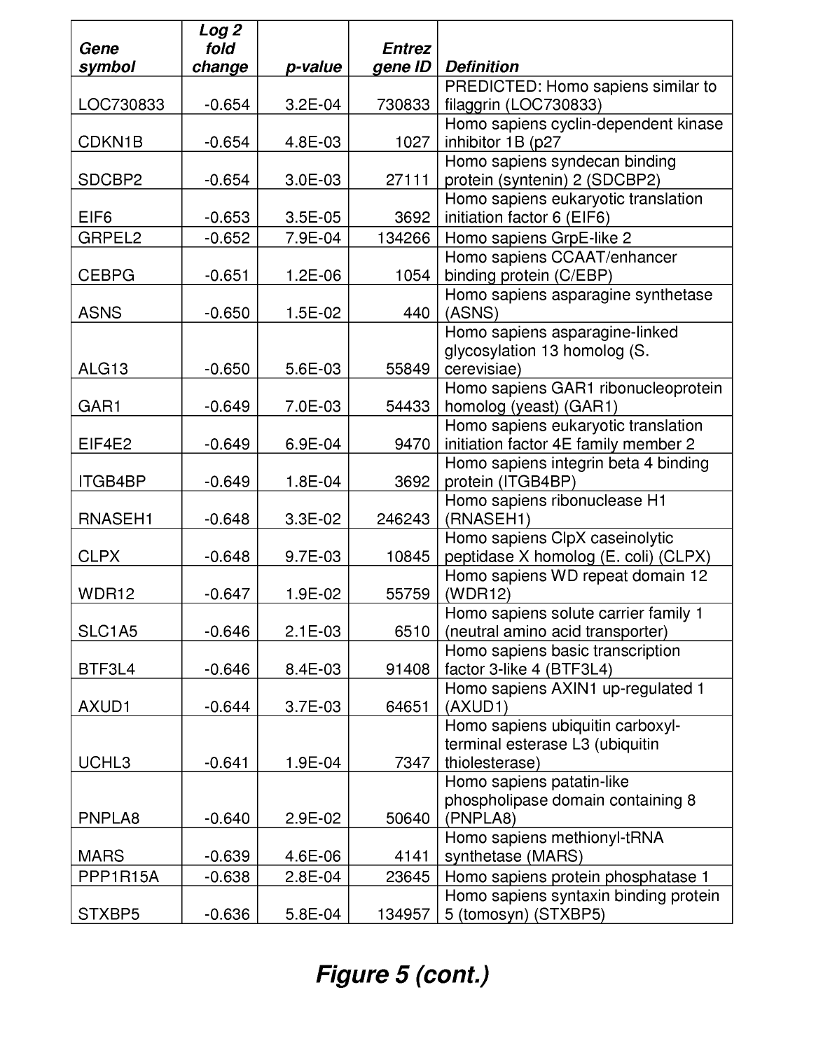

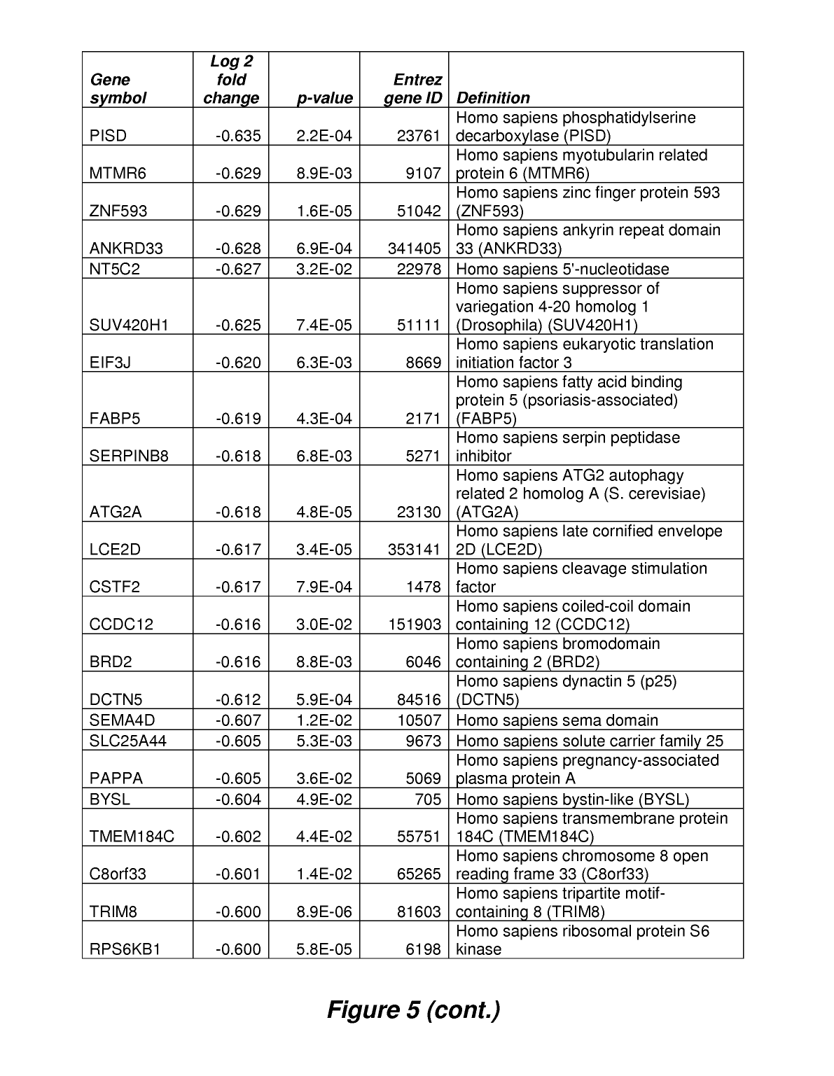

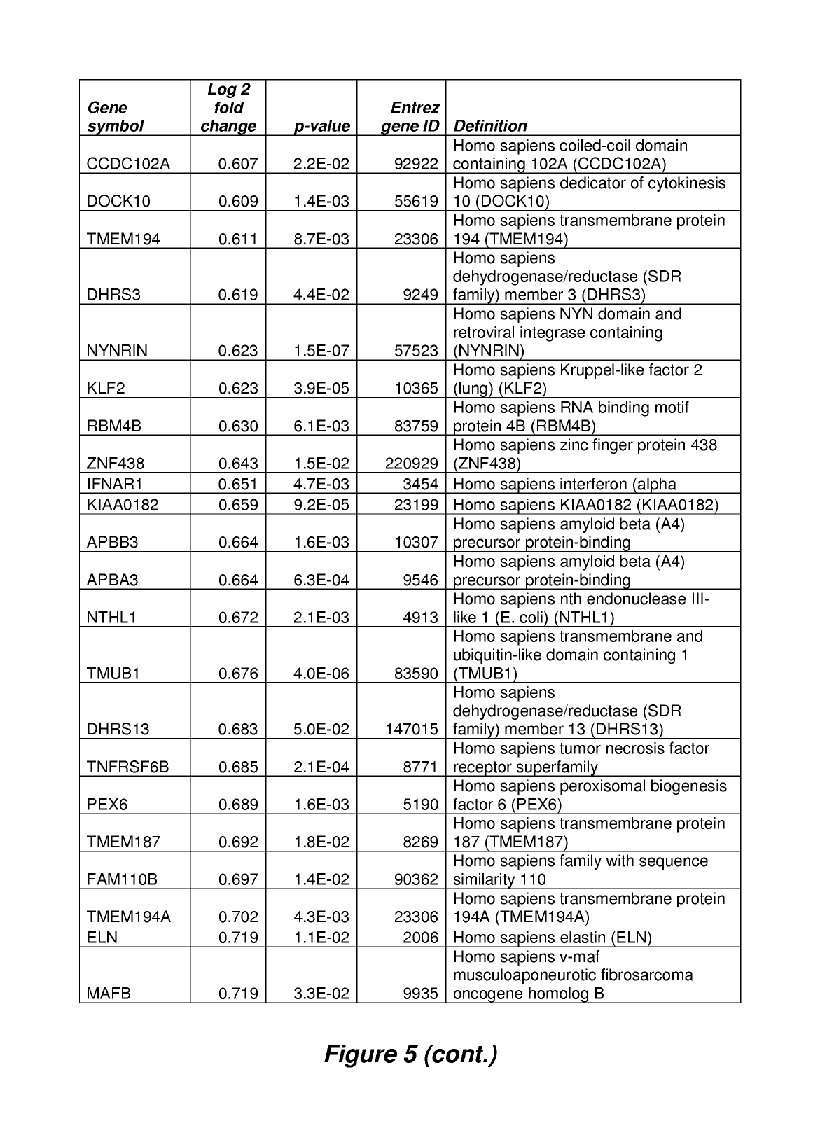

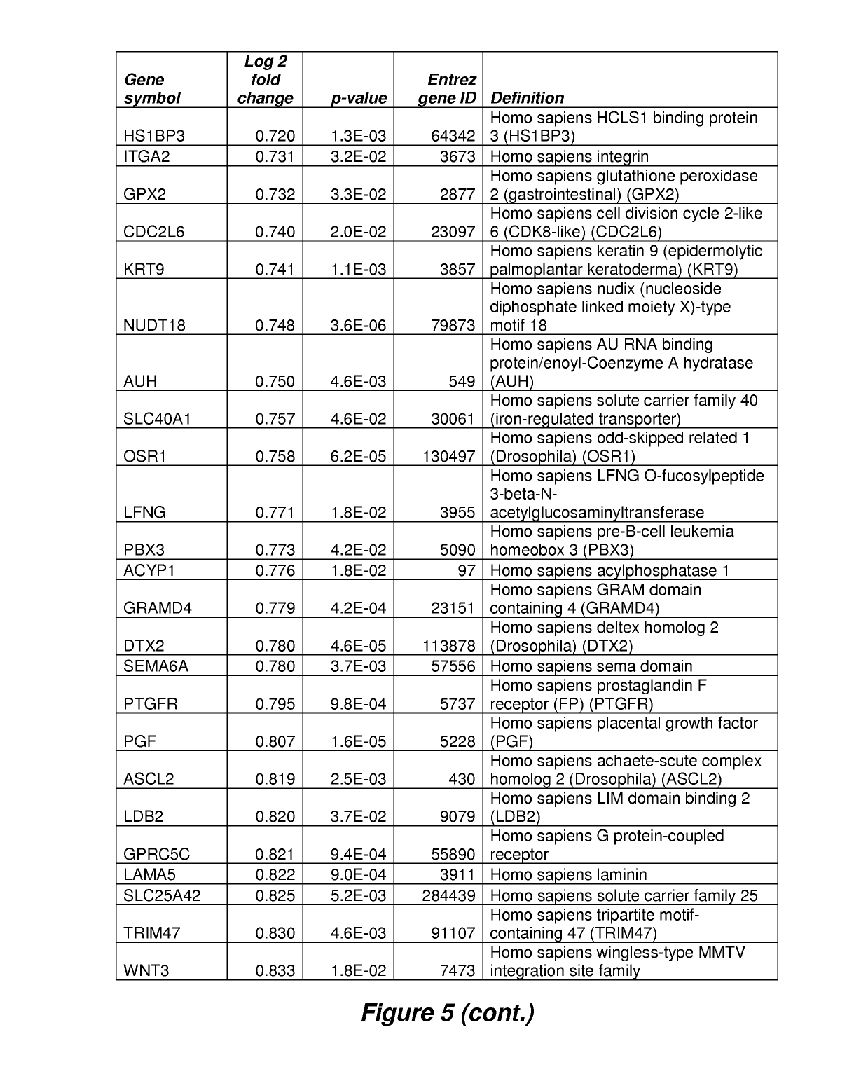

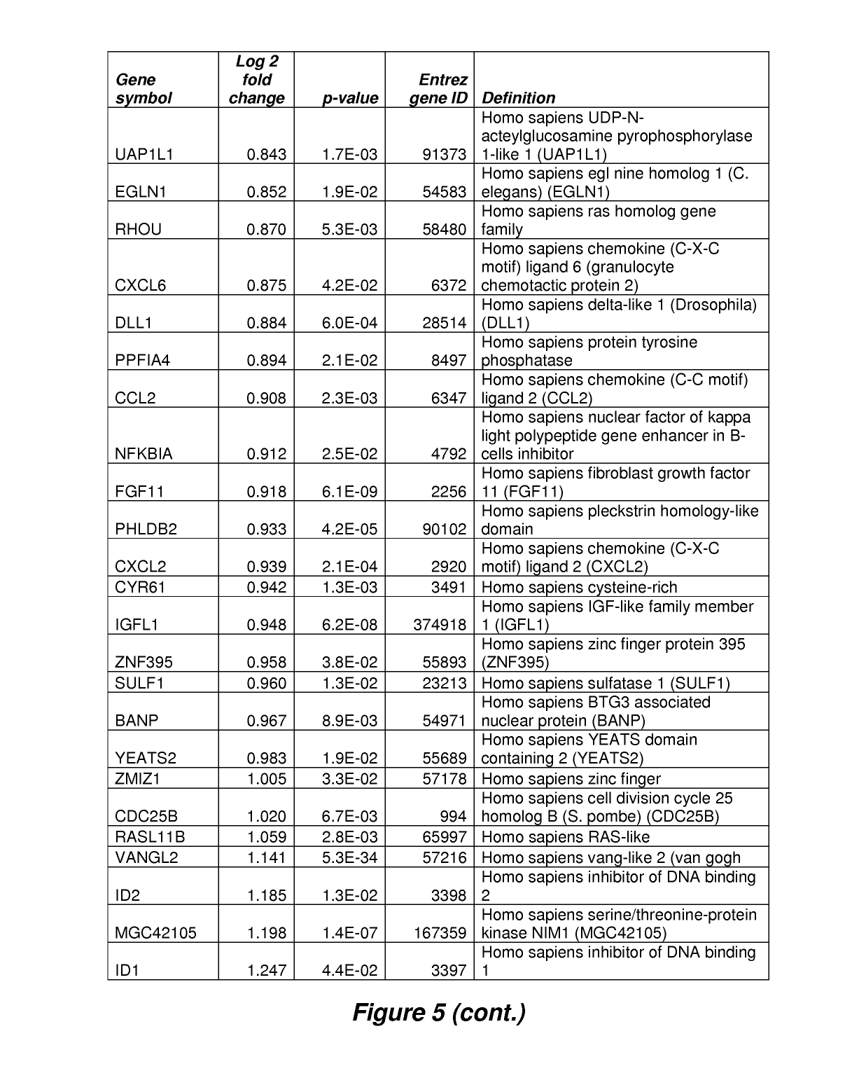

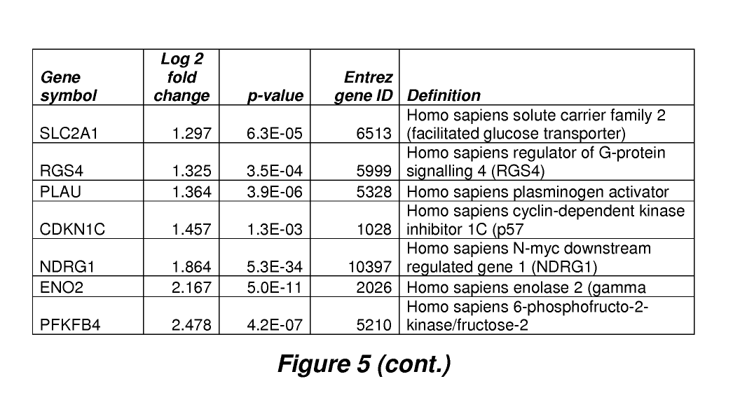

Generally, picosecond THz radiation pulses have been shown to down-regulate genes involved in inflammatory skin disorders and cancers such psoriasis, atopic dermatitis, epidermal hyperplasia, dermatitis, oral squamous cell carcinoma and non-melanoma skin cancer (squamous cell carcinoma and basal cell carcinoma). A non-exhaustive list of genes affected by THz radiation is listed in FIG. 4 and FIG. 5. As used herein, a gene is considered to be up-regulated or down-regulated due to THZ radiation exposure if the gene shows a change in expression levels of at least about 1.5-fold or greater when compared to a control sample. Alternatively in the method, a statistical difference of about two standard deviations or more from the mean is considered significant upon comparing control and test samples.

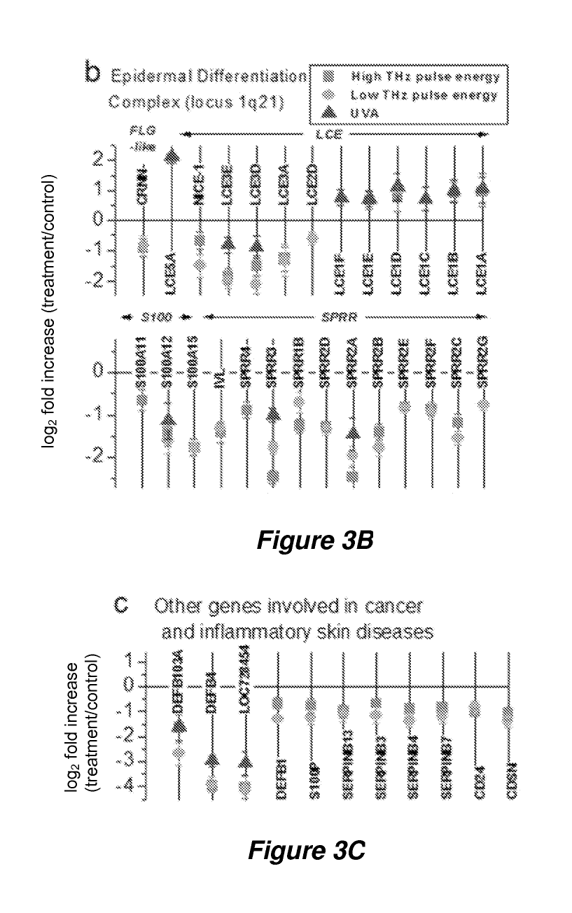

Among those genes down-regulated are members of the Epidermal Differentiation Complex (EDC) genes located on human chromosome 1q21 (see FIG. 3). The EDC comprises 57 genes encoding 5100 proteins as well as structural proteins of epidermal cornification. Many of these genes are up-regulated in skin disorders such psoriasis, atopic dermatitis and skin cancers. These genes include members of the Small Proline Rich Region (SPRR), Late Cornified Envelope (LCE), Involucrin (IVL), NICE-1, Cornulin (CRNN) and S100. At least 16 members of these families, known to be up-regulated in psoriasis, are down-regulated in response to THz radiation. These include, but are not limited to S100A15, S100A12, SPRR2A, SPRR3, SPRR2B, SPRR3, SPRR4, SPRR1B, SPRR2D, SPRR2E, SPRR2F, SPRR2C, LCE3A, LCE2D, LCE3D and LCE3. Other genes down-regulated by THz radiation and implicated in psoriasis are corneodesmosin (CDSN), CD24, members of the human β-defensin family (DEFB103A, DEFB4, LOC728454 and DEFB1), and members of the serine protease inhibitor (serpin) B genes including serpinB3, serpinB4 and serpinB13. Up-regulation of β-defensins is also implicated in basal cell carcinomas.

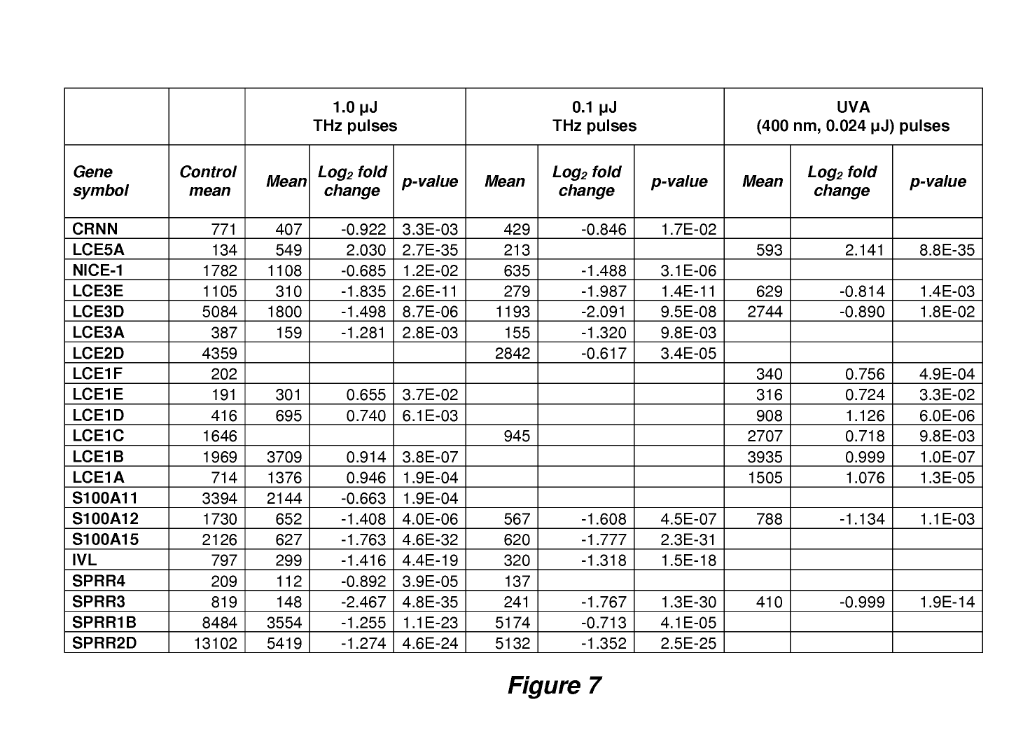

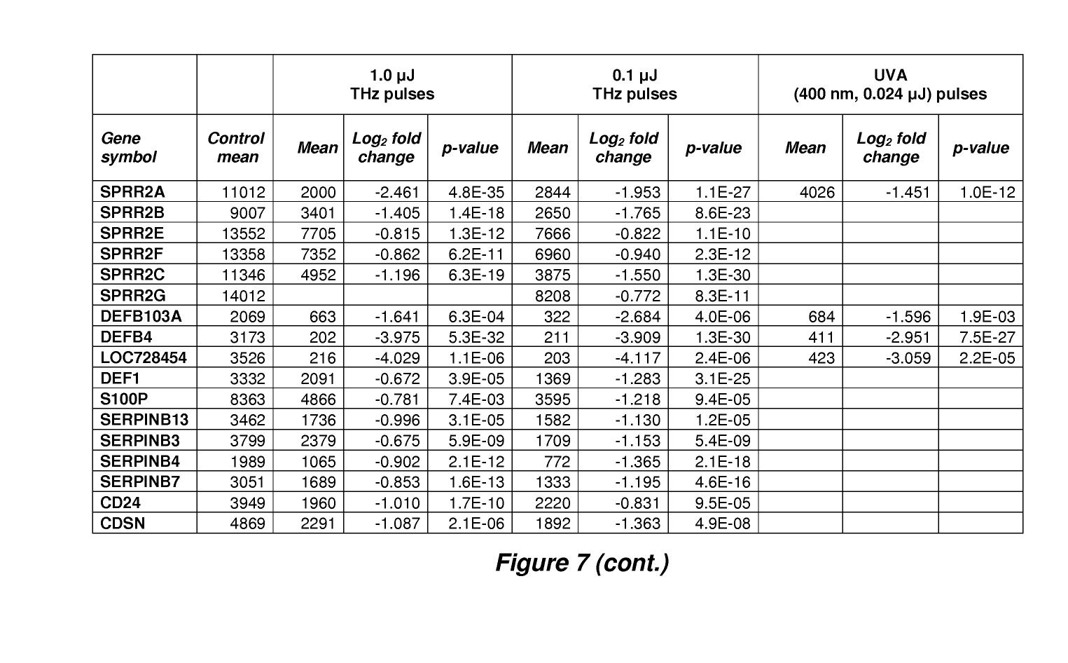

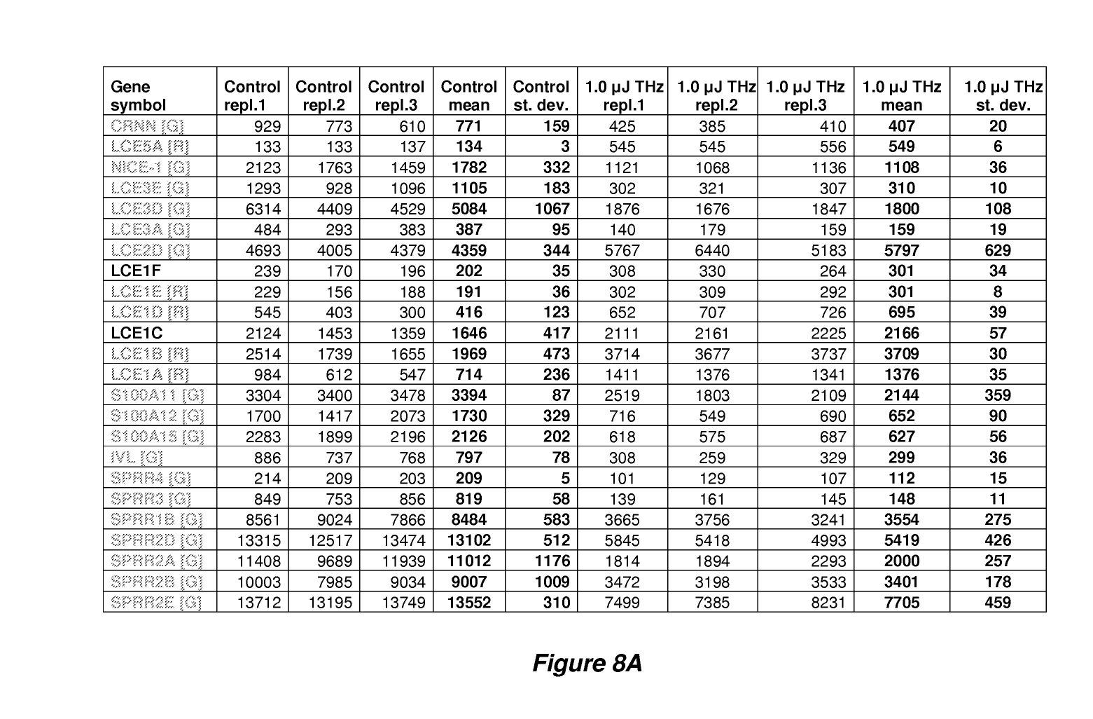

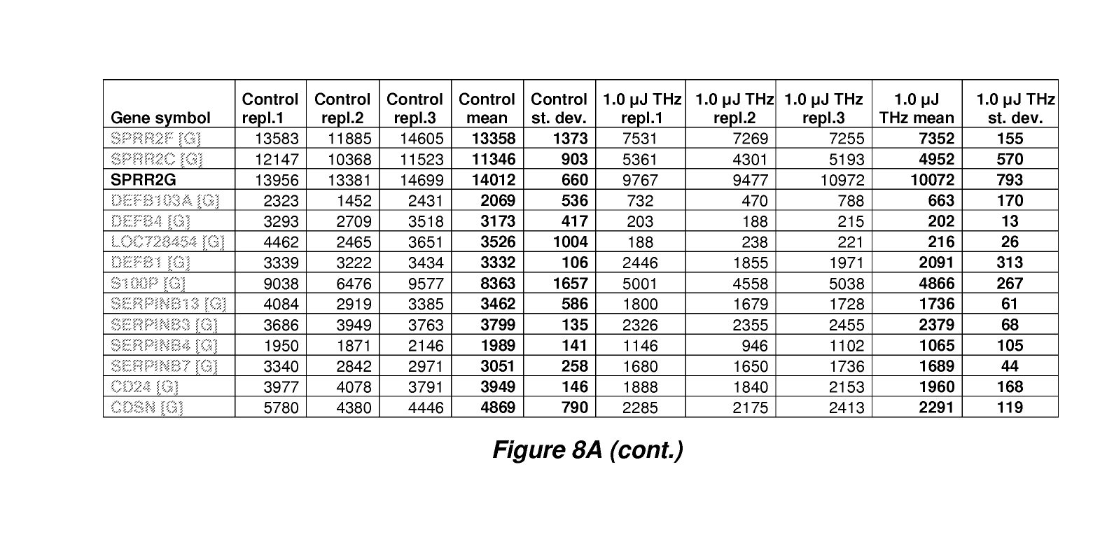

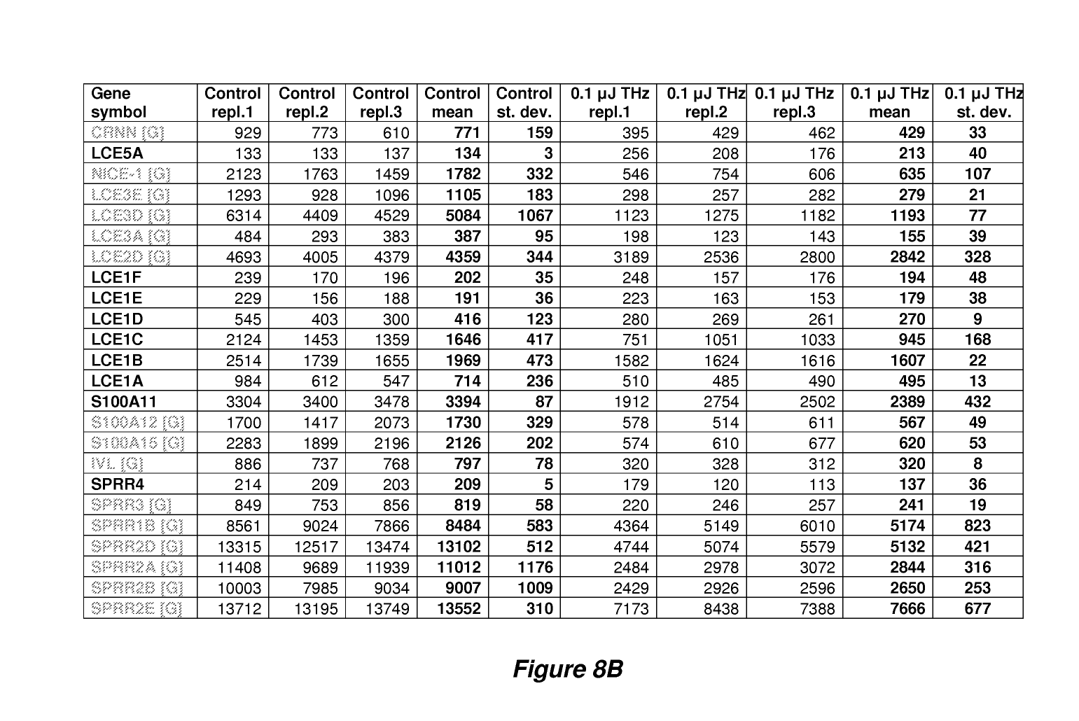

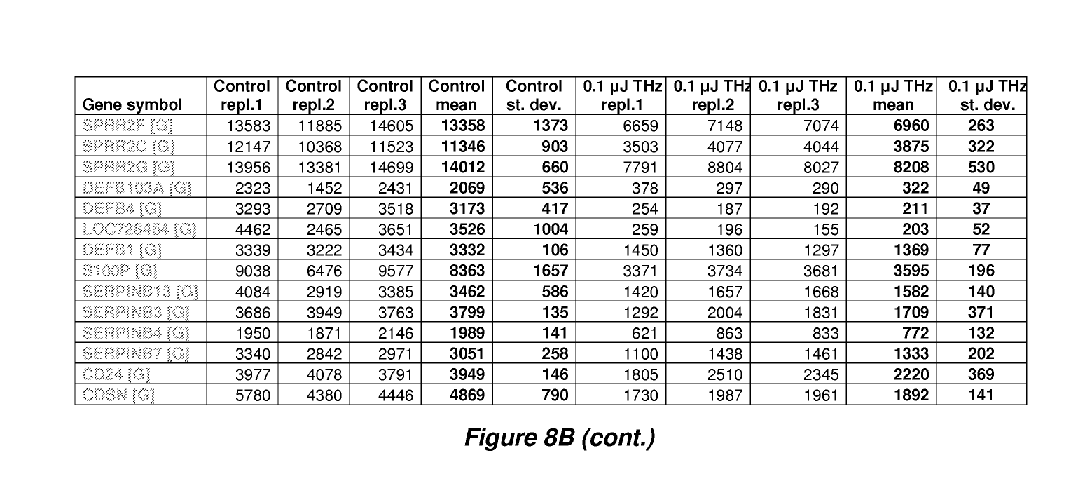

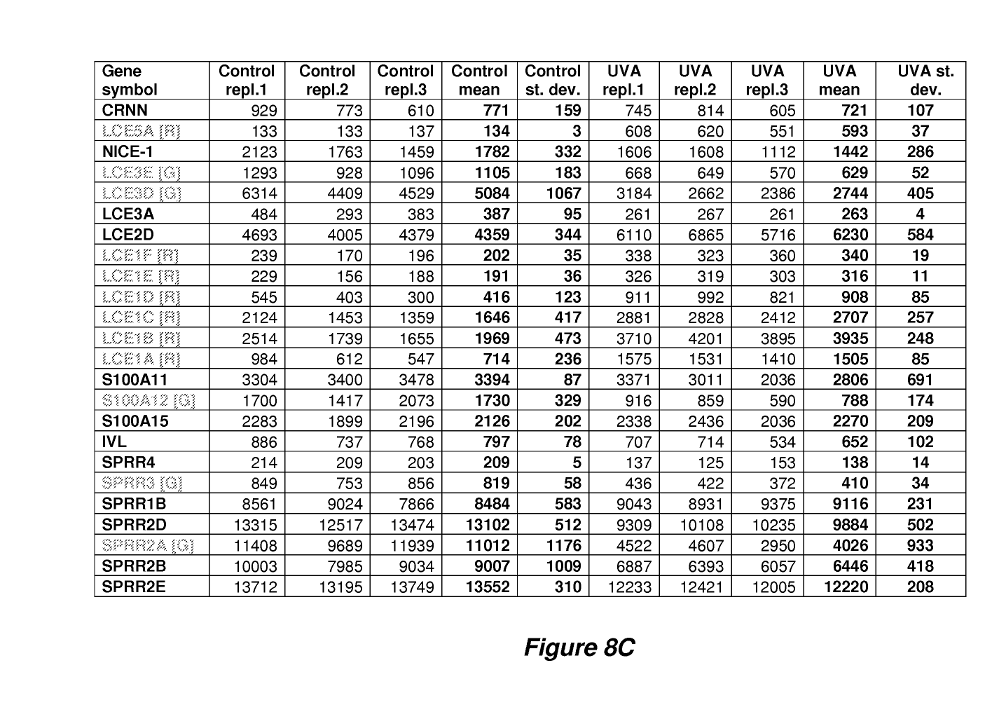

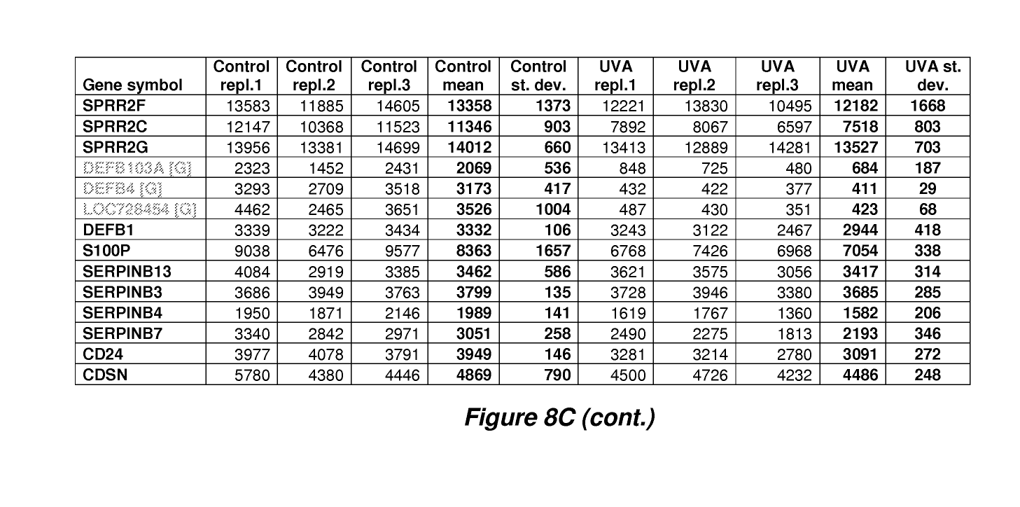

EDC genes, among others, are also up-regulated in cutaneous squamous cell carcinomas. Of these, at least 8 genes are down-regulated by exposure to THz radiation. These include including S100A11, S100A12, SPRR1B, SPRR2B, SPRR2C, SPRR3, IVL, and LCE3D. Other genes down-regulated by THz radiation involved in squamous cell carcinomas include CD24, DEFB103A, DEFB4, LOC728454, DEFB1, serpinB3, serpinB4 and serpinB13 (also see FIG. 7 and FIG. 8).

Furthermore, the EDC gene S100P is known to be up-regulated in many tumor types, acting as a proliferative and anti-apoptotic agent. Moreover, disruption of the S100P gene is known to suppress the growth of hepatocellular carcinoma cells. S100P is down-regulated when exposed to THz radiation.

In some situations, EDC genes may be down-regulated in certain disease states. Some of these genes known to be down-regulated in a certain disease state include, but are not limited to LCE1A, LCE1B, LCE1C, LCE1D, LCE1E, LCE1F and LCE5A. Exposure to THz radiation may result in up-regulation of these genes, leading to a decrease in the disease state (also see FIGS. 4-6 for a list of genes that are up-regulated in response to THz radiation).

In some embodiments, the THz radiation is applied to a human patient. In other embodiments, the THz radiation may be applied to human skin tissue, artificial human skin tissue, or even animal skin tissue to modulate gene expression leading to a reduction, or at least prevent an increase in a disease state.

In addition, THz radiation may modulate genes and proteins sharing a sequence identity or substantial sequence identity to those genes and proteins listed herein.

As used herein, “sequence identity” or “identity” in the context of two nucleic acid or protein sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

As used herein, non-ionizing radiation refers to any type of electromagnetic radiation that does not carry enough energy to completely remove an electron from an atom or molecule, and preferably has photon energy of between about 0.5 meV and about 15 meV.

As used herein, ionizing radiation refers to radiation that carries enough energy to liberate electrons from atoms or molecules, thereby ionizing them and generally consists of gamma rays, X-rays, and the higher ultraviolet part of the electromagnetic spectrum.

As used herein, modulation of gene expression refers to changing the level of expression of a gene at a certain time in response to exposure to THz radiation.

As used herein, THz radiation is refers to electromagnetic waves having band frequencies of 0.1 terahertz to 3 terahertz.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more peptides of a protein refers to one to five, or one to four, for example if the protein is fragmented.

As used herein, the term “about” refers to an approximately +/−10% variation from a given value. It is to be understood that such a variation is always included in any given value provided herein, whether or not it is specifically referred to.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought by those skilled in the art utilizing the teachings of the descriptions herein. It is also understood that such values inherently contain variability necessarily resulting from the standard deviations found in their respective testing measurements.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals, claim elements, and the like.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

Recently, we demonstrated that intense, picosecond THz pulses significantly induce phosphorylation of H2AX in human skin tissue, indicative of DNA damage. We also observed THz-pulse-induced increases in the levels of multiple cell cycle regulatory and tumor suppressor proteins, such as p53, p21, p16, Ku70, and EGR1, suggesting that cellular DNA repair machinery is activated in response to THz-pulse-induced DNA damage. Furthermore, recent experiments have demonstrated that exposure to broadband, picosecond-duration THz pulses results in gene expression changes that depend on the type of exposed cells or tissues and are non-thermal in nature. For example, broadband, picosecond-duration THz pulses influenced differentiation of mammalian stem cells and elicited parameter-specific changes in the expression levels of genes that were transcriptionally active at a given differentiation time point. In human skin tissue, it was observed that intense THz pulses caused changes in expression levels of several cancer-related genes.

Since the penetration of THz radiation into the human body is limited to the sub-mm range, its effects on skin tissue are most relevant for potential clinical applications. In our preliminary study of the effects of intense THz pulses on human skin tissue, we analyzed gene expression in the dissected portions of the tissue containing both directly exposed as well as neighboring unexposed portions, and noted changes in gene expression. Yet, it is important to precisely delineate the changes that occur exclusively in THz-exposed tissue. Here we report on the impact of intense THz pulses on the global gene expression profile in human skin tissue. We find that exposure to intense THz pulses results in coordinated changes in expression of multiple genes involved in epidermal differentiation. Many of the affected genes are implicated in inflammatory skin diseases such as psoriasis and atopic dermatitis and in non-melanoma skin cancer, suggesting the possibility for novel treatment modalities based on intense THz pulses.

### Results

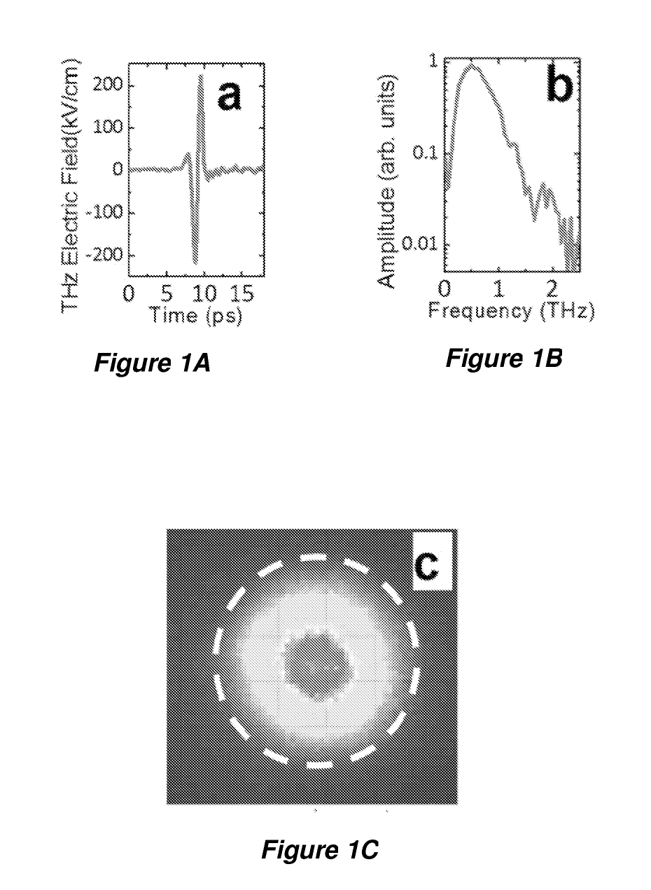

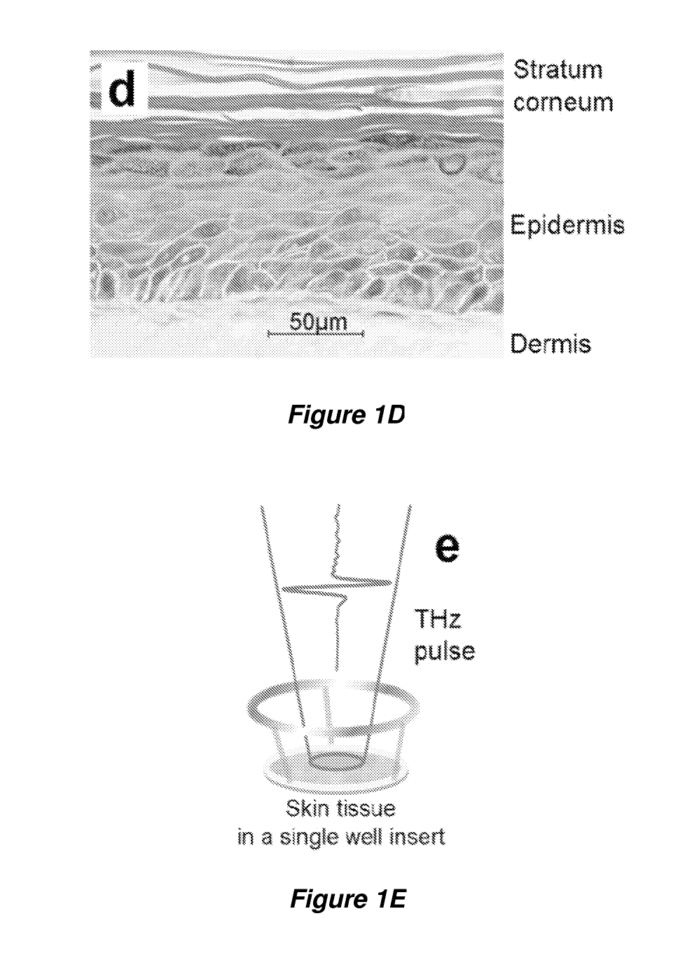

We have exposed artificial human skin tissues to picosecond-duration (FIG. 1a) broadband (0.2-2.5 THz, FIG. 1b) THz pulses with 1 kHz repetition rate, 1/e² spot-size diameter of 1.5 mm (FIG. 1c) and pulse energies of either 1.0 μJ or 0.1 μJ. For comparison, we have exposed skin tissues to UVA pulses (400 nm, 0.1 ps, 0.024 μJ). While DNA does not directly absorb UVA, its cytotoxicity and genotoxicity are mediated by the generation of reactive oxygen species that in turn damage DNA and other cellular components.

The full thickness human normal skin tissue used, EpiDermFT, is a multilayered, highly differentiated model of dermis and epidermis (FIG. 1d). It is mitotically and metabolically active, preserves the arrangement and communication of cells present in tissues in vivo, and thus provides an excellent platform for assessing the effects of exposure to intense THz pulses. Tissue samples were placed at the focus of the pulsed THz beam for 10 minutes (FIG. 1e) or at the focus of the pulsed 400 nm beam for two minutes. Global gene expression in excised 2 mm-diameter exposed portions of the tissues (as illustrated schematically by a dashed circle in FIG. 1d) was analyzed using an Illumina HumanHT-12 v4 Expression BeadChip 30 minutes after irradiation.

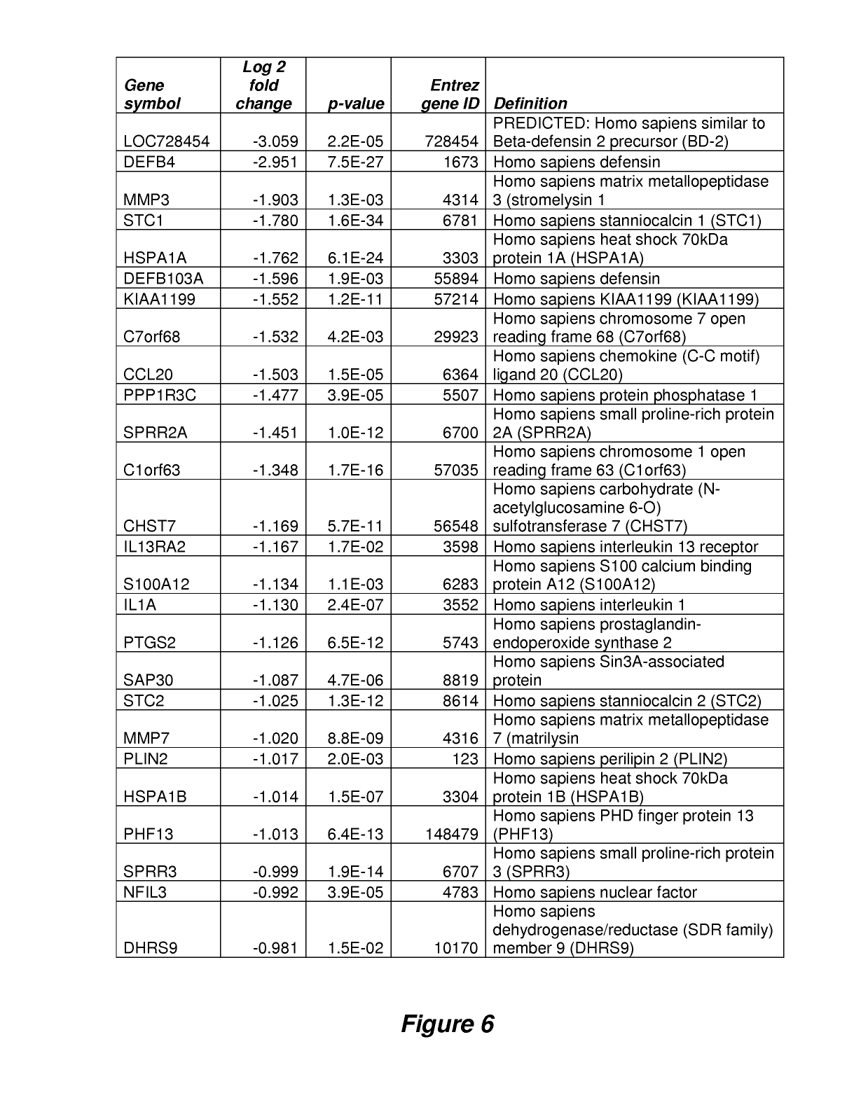

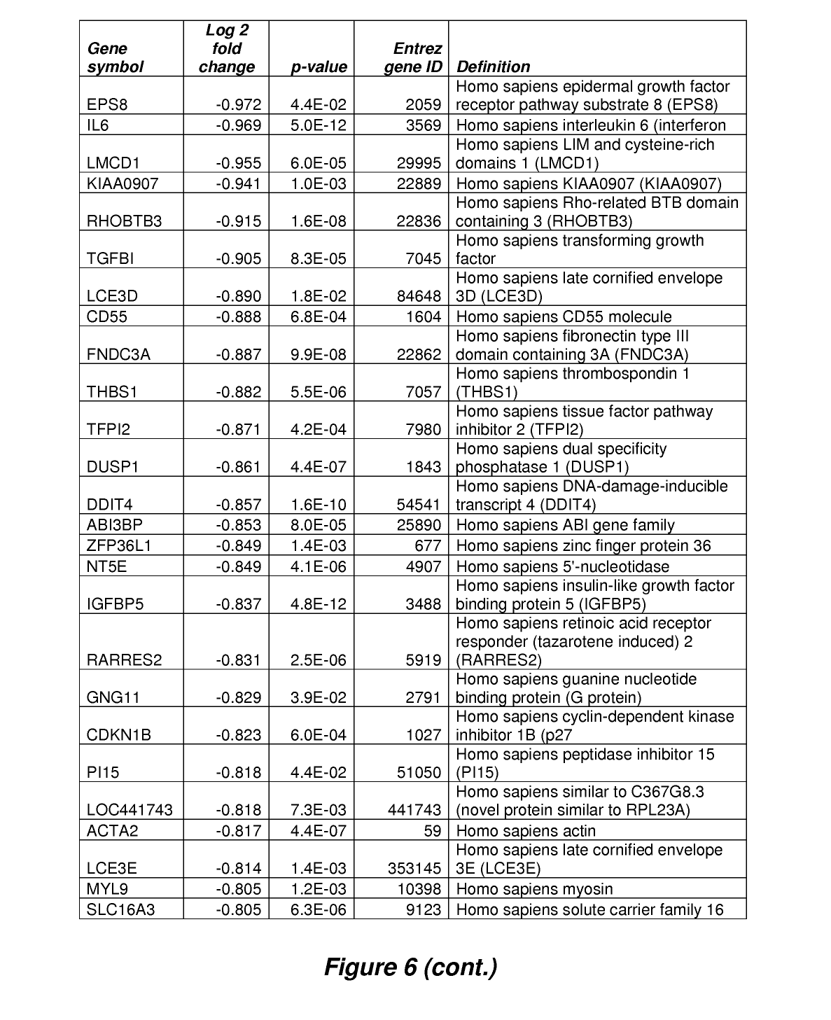

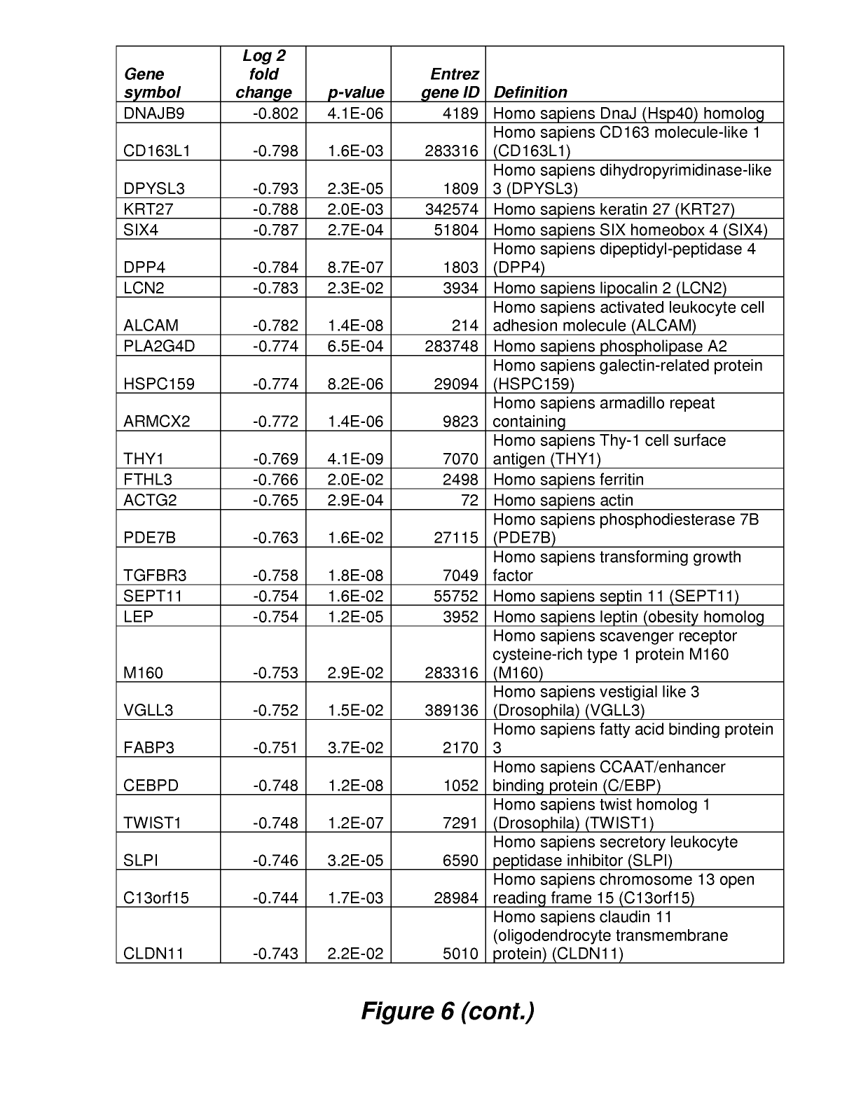

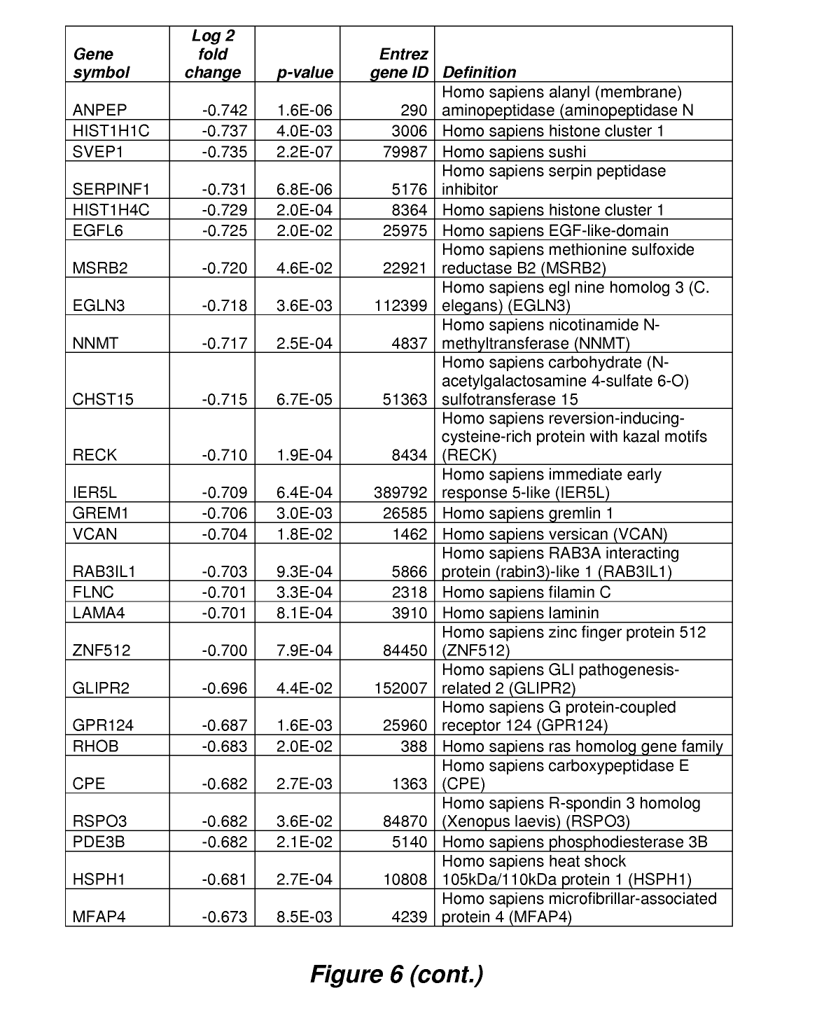

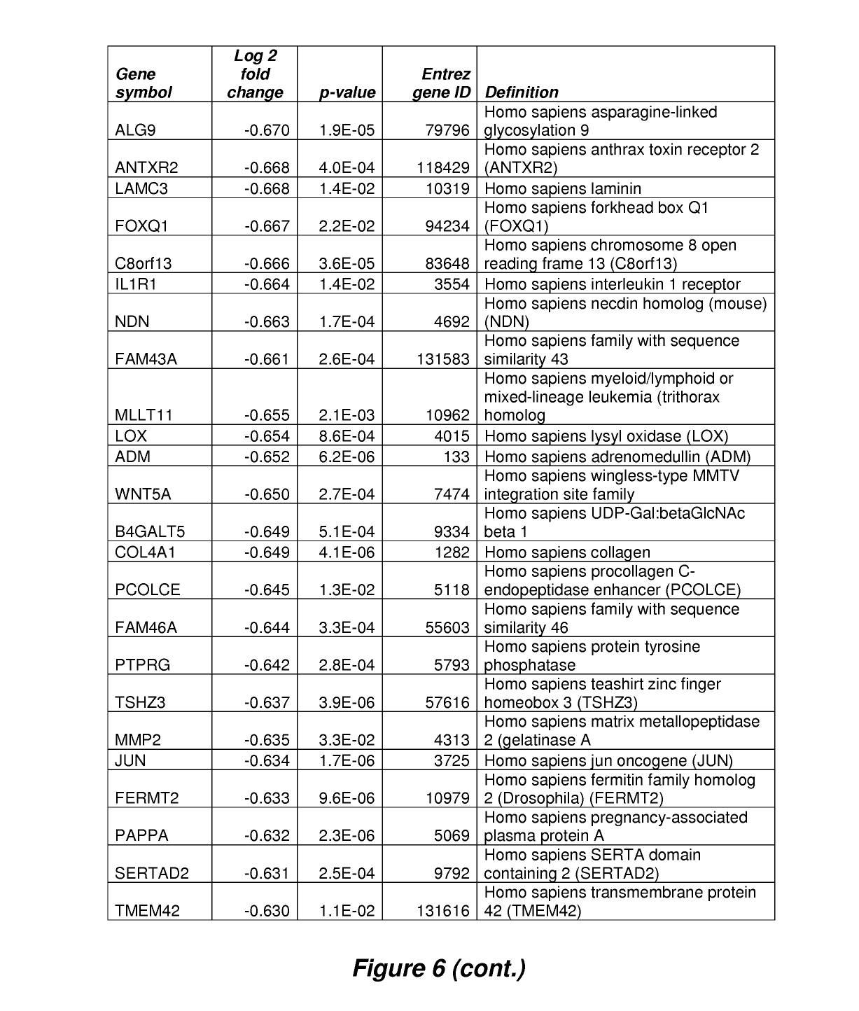

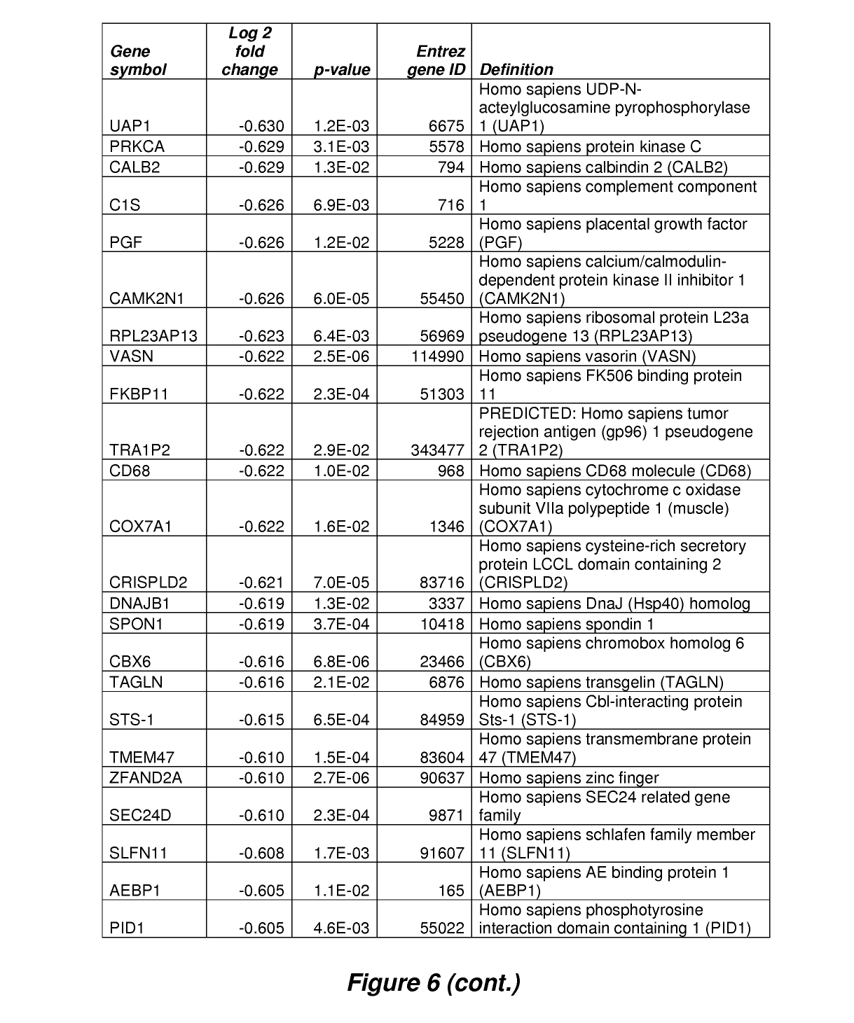

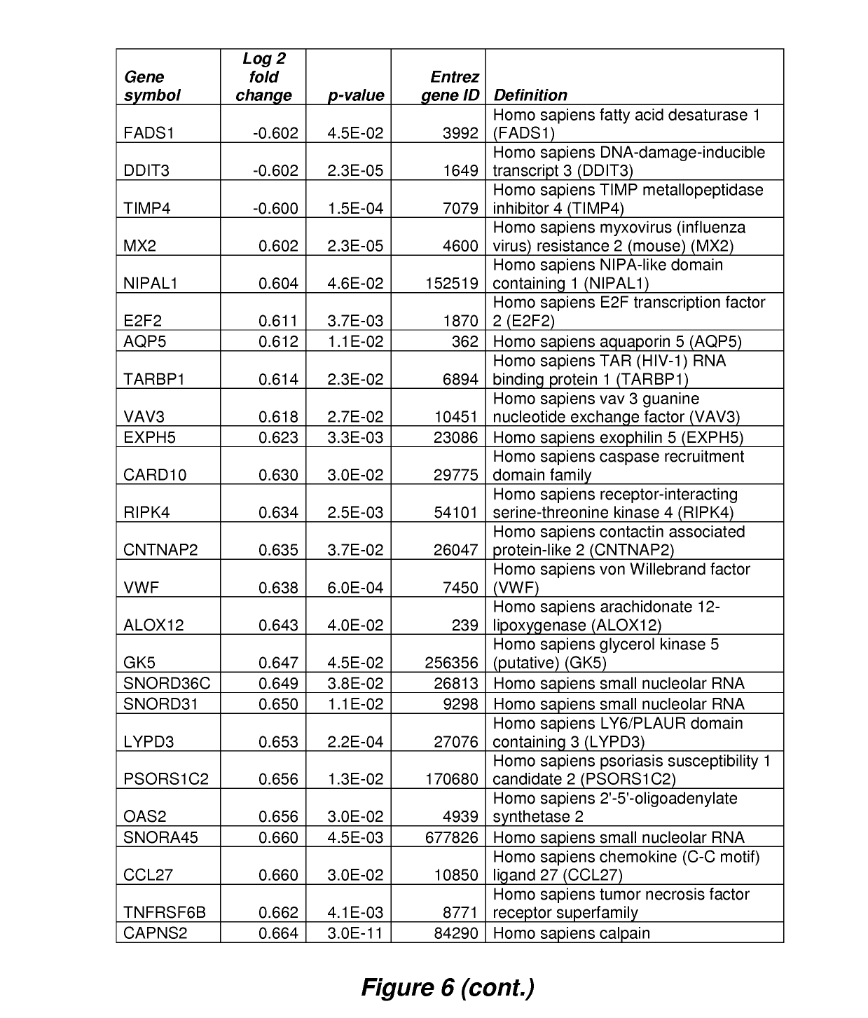

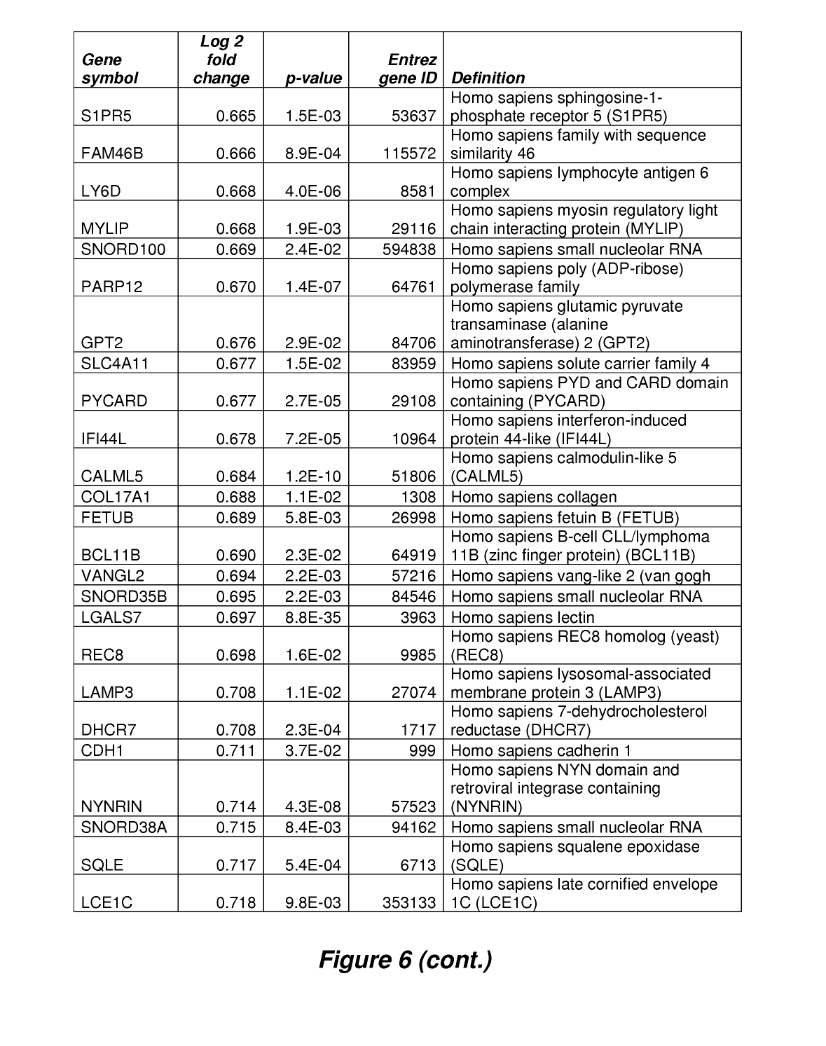

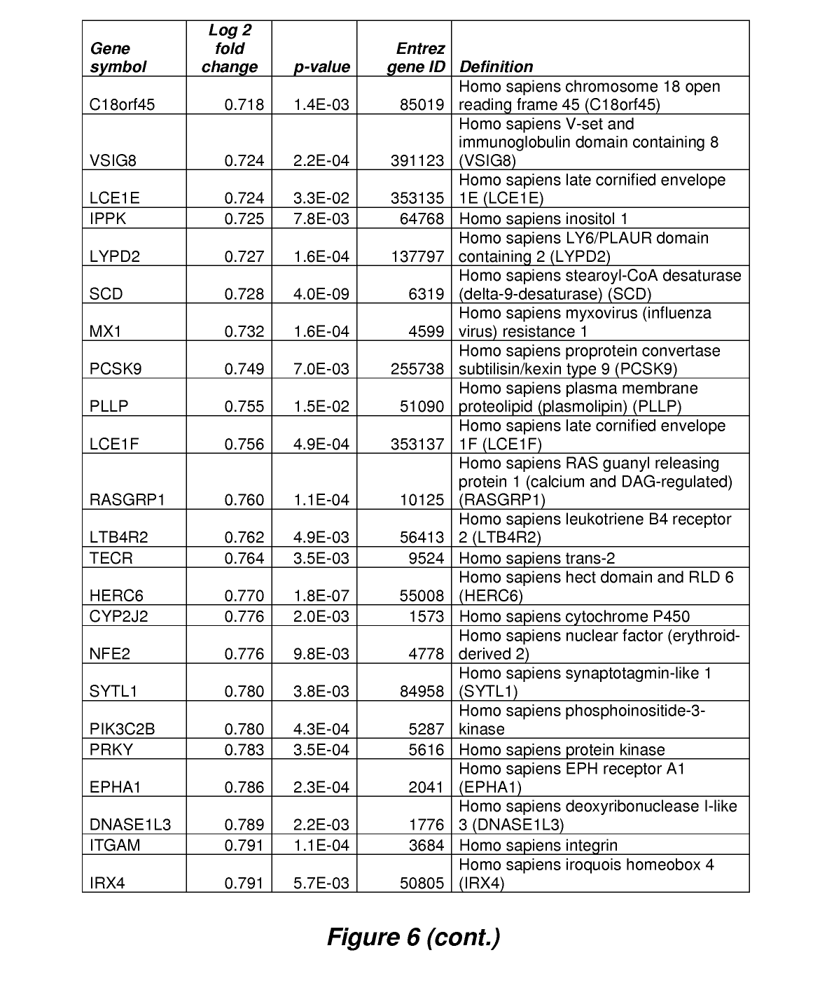

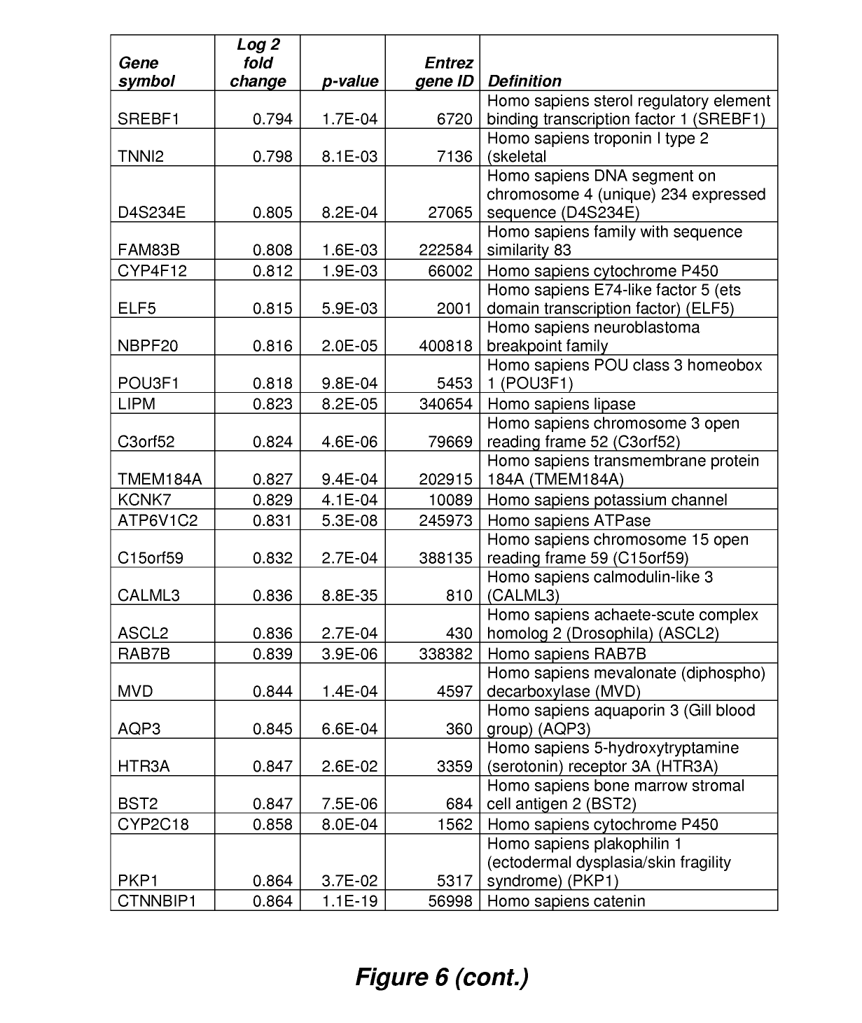

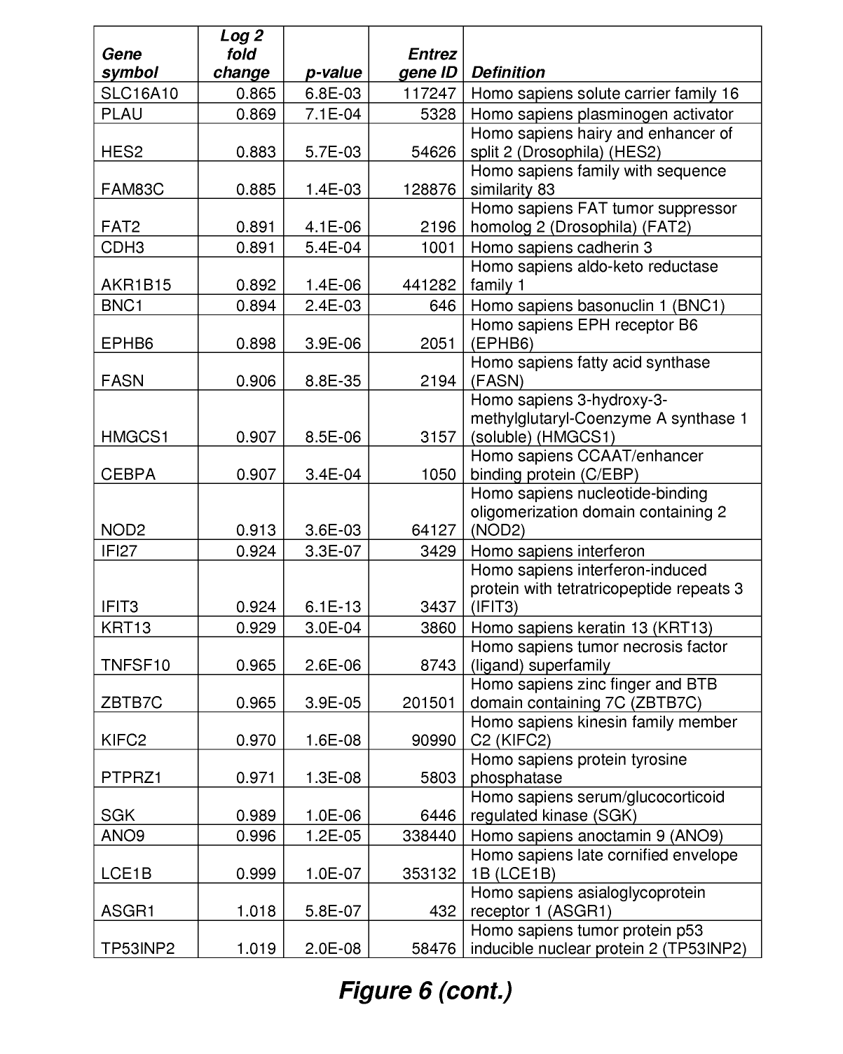

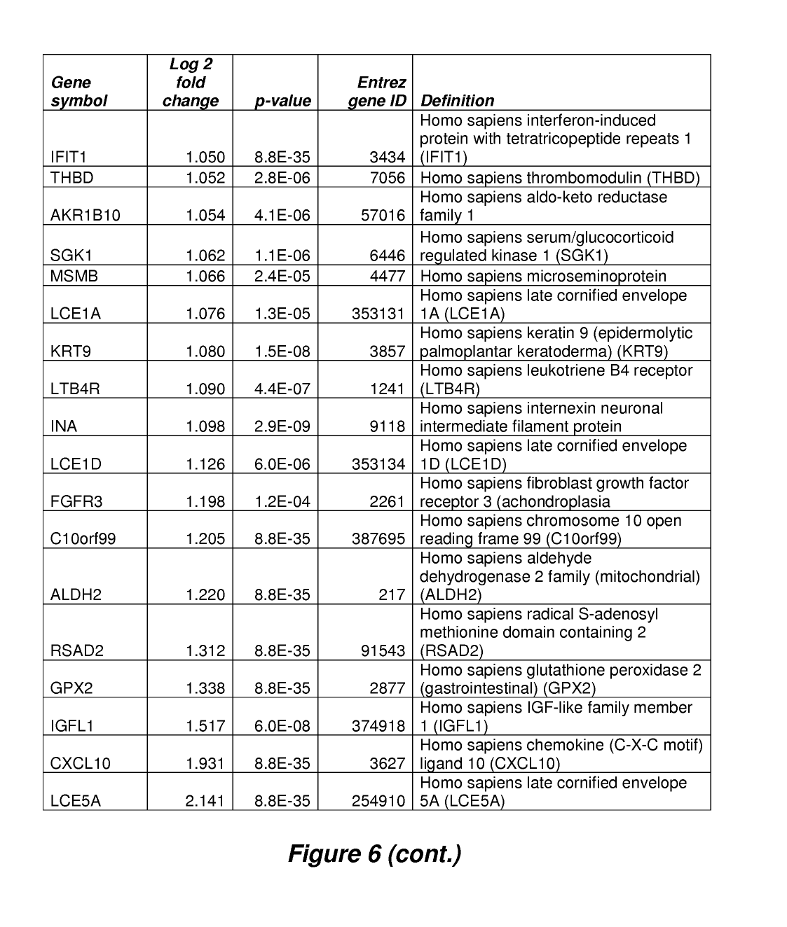

We found that intense THz pulses profoundly affect gene expression in directly exposed human skin. Exposure to THz pulses with an energy of 1.0 μJ for 10 minutes altered the levels of 442 genes (FIG. 4), while irradiation by pulses with 10 times lower energy (0.1 μJ) for the same time duration affected the expression of 397 genes as compared to unexposed controls (FIG. 5). Interestingly, exposure to UVA pulses caused changes in the expression of only 290 genes (FIG. 6). The observed changes in gene expression profile were not caused by THz-induced tissue heating. THz exposures were carried out at ambient temperature (21° C.), and the time-averaged power (1 mW) was low. Applying a theoretical formalism developed in ref 20, we estimate that the temperature increase due to THz exposure was less than 0.7° C. Furthermore, none of the heat shock protein encoding genes were differentially expressed in THz- or UVA-exposed tissues.

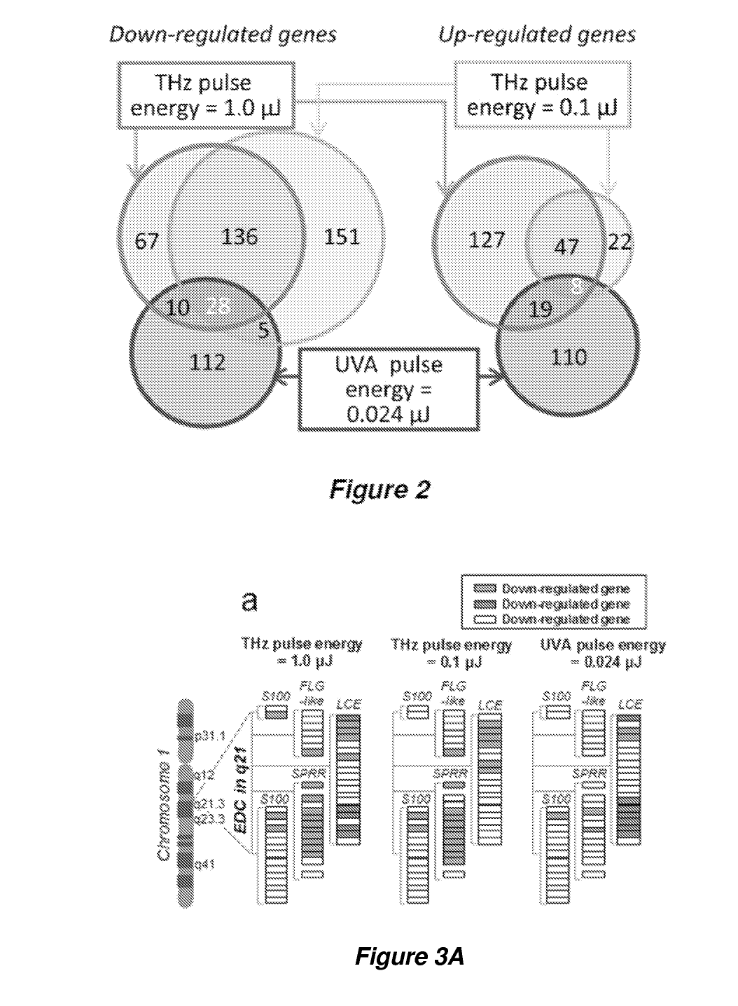

Analysis of the overlaps in the lists of genes differentially expressed upon exposure to intense pulsed THz and UV radiation revealed interesting trends (FIG. 2). While there is a significant overlap between genes affected by both THz pulse energies, little overlap exists between THz- and UVA-induced gene expression profiles, which is not surprising since there is a fundamental difference in how low photon energy (˜4 meV), non-ionizing THz radiation and high photon energy (3.1 eV) UVA radiation interact with living cells.

Differentially-expressed genes that were common for both THz pulse exposure regimes are of particular interest as potential THz-pulse-exposure biomarkers that hold the key to understanding cellular effects of intense THz pulses. We identified 219 such genes, of which 164 are down-regulated and 55 are up-regulated as a result of THz pulse exposure. Among them are genes involved in molecular etiology of dermatological diseases (psoriasis, atopic dermatitis, epidermal hyperplasia, and dermatitis), cancer, and genes with important functions in apoptotic signaling pathways.

We also find that the affected genes are not uniformly distributed throughout the genome. The most striking changes were observed in the expression of epidermal differentiation complex (EDC) genes. EDC describes the 1.6-Mb locus on human chromosome 1q21 that contains 57 genes encoding S100 proteins as well as structural proteins of epidermal cornification (FIG. 3a). Of them, 16 were markedly down-regulated by exposure to intense THz pulses in both intensity regimes, compared to only 5 affected by UVA pulses (FIG. 3b). THz-pulse-affected genes include members of the Small Proline Rich Region (SPRR), involucrin (IVL), Late Cornified Envelope (LCE) and the S100 families, as well as NICE-1 and comulin (CRNN). In addition to EDC genes, exposure to intense THz pulses also altered expression levels of many other genes implicated in cancer and dermatological diseases (FIG. 3e).

### Discussion.

EDC complex genes are responsible for terminal differentiation of keratinocytes and regulation of epidermal barrier function, as well as for skin immune and inflammation responses, Up-regulation of EDC genes leads to increased proliferation and differentiation of keratinocytes commonly observed in inflammatory skin disorders such as psoriasis and atopic dermatitis, as well as in skin cancers. The 1q21 locus has been identified as one of the key psoriasis susceptibility loci (PSORS4) in clinical genome-wide linkage studies. Of the EDC genes that were down-regulated by intense THz pulses (FIG. 3b), many, including S100A15, S100A12, SPRR2A, SPRR3, SPRR2B, LCE3 genes, are up-regulated in psoriasis.

Enhanced expression of multiple EDC genes, including members of the SPRR family, S100 genes that encode for calcium-binding proteins, and multiple cornified envelope genes, has been identified as a hallmark of cutaneous squamous cell carcinomas (SCC). Of these SCC-associated EDC genes, eight, including S100A11, S100A12, SPRR1B, SPRR2B, SPRR2C, SPRR3, involucrin (IVL), and LCE3D, are down-regulated by intense THz pulse exposure (FIG. 3b). Down regulation of these genes by intense THz pulses may open new avenues for targeted treatments for psoriasis and SCC.

Besides EDC genes, THz pulses affected numerous other genes involved in psoriasis, atopic dermatitis and other inflammatory skin diseases, as well as in many cancer types, such as aggressive oral squamous cell carcinoma (OSCC) and non-melanoma skin cancer (SCC and basal cell carcinoma (BCC)) (FIG. 3c). We observe down regulation of corneodesmosin (CDSN), a member of the PSORS1 psoriasis susceptibility locus, and CD24, a key pro-inflammatory gene, both of which are usually over-expressed in psoriatic skin. The CD24 gene encodes for a mucin-like adhesion molecule, and its over expression has been also repeatedly associated with aggressive tumor progression, metastasis and poor prognosis in various cancers. It is also up-regulated in SCC. Additionally, THz exposure significantly lowered mRNA expression of human β-defensins (DEFB103A, DEFB4, LOC728454, and DEFB1). Interestingly, the impact of intense THz pulses on the expression levels of β-defensins was similar to that of UVA pulses. Enhanced expression of β-defensins is associated with non-melanoma skin cancer (basal cell carcinoma (BCC) and SCC), and OSCC, as well as psoriasis.

Exposure to intense THz pulses also suppressed levels of four serine proteinase inhibitor (serpin) B genes that map to the serpin superfamily locus at chromosome 18₈21.3, once again demonstrating a non-uniform distribution of intense-THz-pulse-affected genes throughout the genome. Two of them, serpinB3 and serpinB4, are also known as squamous cell carcinoma antigens 1 and 2, respectively. Their elevated expression is associated with aggressive SCCs and psoriasis. SerpinB13, or hurpin, is also over-expressed in SCC, as well as in psoriatic lesions. Another down-regulated member of the serpin family is serpinB7, or megsin. Finally, intense THz pulses (but not UVA pulses) down-regulated the S100P gene that is related in function to the S100A family of genes of EDC and encodes for a member of the Ca²⁺-binding proteins family. Over-expression of S100P has been shown to act as a proliferative and anti-apoptotic factor in many tumor types. Moreover, targeted disruption of S100P was recently shown to suppress the growth of hepatocellullar carcinoma cells. While the exact role of serpins as well as S100P in skin cancer and inflammatory skin diseases is yet to be elucidated, their concerted down-regulation by intense THz pulses might have clinical relevance and should therefore be further investigated.

Furthermore, the mechanisms by which intense THz pulses influence gene expression are not yet known. Based on mesoscopic modelling of DNA breathing dynamics in a THz field, it has been suggested that THz radiation may amplify existing (or create new) open states in the double helix, thereby affecting transcription initiation or binding of transcription factors. Alternatively, the changes in gene expression may constitute a cellular response to intense-THz-pulse-induced damage to DNA or intracellular proteins, which initiates multiple cellular-damage-repair pathways. In the future, a detailed analysis of the impact of intense THz pulses on the activity of various transcription factors common to affected genes is needed to elucidate the exact cellular and molecular effects of intense THz pulses.

Additionally, analysis of transcriptional changes seen in skin tissue that is directly exposed to intense THz radiation and in the mixture of directly exposed and neighboring naïve tissue′ indicates that exposure to intense THz pulses can induce out-of-field bystander effects. While bystander effects are accepted to occur after exposure to ionizing radiation, the existence, extent and mechanisms of THz-pulse-induced bystander effects need to be further analyzed.

While a number of earlier studies investigated the biological effects of continuous-wave THz radiation and found that the majority of the observed effects are thermal in nature, the present study is one of the very few to date that have explored genotoxic, cytotoxic and epigenetic effects of picosecond and subpicosecond broadband THz pulses. The low average power of THz pulse sources results in biologically insignificant temperature increases of only fractions of a degree at most. However, the high energy density per pulse produces peak powers and corresponding electric fields that can be extremely high. It is likely that these high peak electric fields are responsible for the observed THz-pulse-driven cellular effects.

In 2003, Clothier and Bourne reported that picosecond-duration, 0.2-3.0 THz bandwidth THz pulses had no discernible effect on the differentiation, activity or viability of primary human keratinocytes in vitro. Based on the experimental details provided in the paper, we estimate that the energy density per pulse was ˜3 pJ/cm² (3×10⁻⁶ μJ/cm²). In a recent work, Williams et al. explored the influence of more intense THz pulses produced by the ALICE (Daresbury Laboratory, UK) synchrotron source, with energy densities per pulse reaching 10 nJ/cm² (0.01 μJ/cm²), on the attachment, morphology, proliferation and differentiation of human epithelial and embryonic stem cells. Like Clothier and Bourne, they reported no changes in cell properties.

On the other hand, a series of experiments investigating the effects of broadband (˜1-15 THz, centered at 10 THz), subpicosend THz pulses on mouse mesenchymal stem cells (Bock et al., Alexandrov et al.) revealed THz-induced changes in the expression of the genes transcriptionally active at a given differentiation stage of stem cells. Furthermore, the same studies found that THz pulse exposure accelerated cell differentiation toward adipose phenotype. In these experiments, the energy density per pulse was significantly higher at 1 μJ/cm², which may suggest the existence of a THz pulse energy density (and corresponding peak THz electric field) threshold for the induction of changes in cellular function. Finally, in our work, the energy density per pulse was ˜6 μJ/cm² for 0.1 μJ THz pulses and ˜60 μJ/cm² for 1 μJ THz pulses, resulting in specific gene expression changes in human skin tissue. Clearly, more extensive studies are needed to determine pulse energy density thresholds and other exposure parameters for the onset of THz-pulse-induced changes to gene expression and cellular function.

In summary, we have shown that intense THz pulses have profound impact on global gene expression in human skin. Furthermore, we observed that the distribution of intense-THz-pulse-sensitive genes is not uniform across the genome, with a significant number of affected genes belonging to the epidermal differentiation complex in the 1q21 locus. Finally, the observed THz-induced changes in expression of EDC genes and other genes implicated in non-melanoma skin cancers and inflammatory skin disorders such as psoriasis, suggests the potential application of intense THz pulses for treatment aimed at normalizing the expression of these disease-related genes. Picosecond duration, intense THz pulses are non-ionizing, do not cause tissue overheating and can be focused with ˜1 mm precision for delivering safe, non-invasive treatment. Of course, since the present study concentrated on analyzing changes in gene expression in human skin tissue 30 minutes after 10-minute-long exposure to intense THz pulses, it essentially provides a snapshot of THz-pulse-induced effects. Extensive studies tracking the THz-induced-changes in gene expression and the resulting changes in protein expression over a period of time following the exposure of healthy as well as diseased tissues will have to be carried out to assess the full potential of intense THz pulses for therapeutic applications. We envision the study presented here as an essential roadmap for this future work.

### Materials and Methods.

Generation and Characterization of Intense THz Pulses.

We have exposed human skin tissues to THz pulses with 1 kHz repetition rate and pulse energies of either 1.0 μJ or 0.1 μJ. The THz pulses were generated by optical rectification of tilted-pulse-front 800 nm pulses from an amplified Ti:sapphire laser source in a LiNbO₃ crystal. A black polyethylene sheet placed after the LiNbO₃ crystal filtered out the remaining 800 nm beam while letting the THz pulses through with minimal attenuation. The energy of the THz pulses was monitored by a pyroelectric detector (Spectrum Detector, Inc.) with an active area of 6×7 mm². Wire grid polarizers were used to attenuate the THz pulse energy. The THz pulse waveform (FIG. 1a) was measured by electro-optic sampling in 0.5 mm-thick [110] ZnTe crystal. The THz spot size at the focus was determined by imaging the THz beam on a pyroelectric infrared camera. The peak electric field was calculated as discussed. The peak THz electric field was 220 kV/cm for 1.0 μJ and 70 kV/cm for 0.1 μJ energy THz pulses. Exposure to 400 nm pulses for two minutes was also used. The UVA pulses were generated by second harmonic generation in a BBO crystal. A shortpass filter placed after the BBO crystal blocked any remaining 800 nm beam. The UVA spot size at the tissue surface was 2.7 mm diameter.

Tissue Models and Handling.

We used the in vivo full thickness artificial human skin tissues model, EpiDermFT by MatTek, to study the effects of intense THz pulses on gene expression. EpiDermFT recreates normal skin tissue structure with differentiated dermis and epidermis. It consists of human-derived epidermal keratinocytes and dermal fibroblasts that are mitotically and metabolically active. The tissues were cultured according to the manufacturer's protocol, using an air-liquid interface tissue culture technique. Unexposed tissue samples served as controls. Skin tissues in single well plate inserts were placed at the focus of the THz beam for 10 minutes, or at the focus of the UVA beam for 2 minutes.

Four tissue samples were used for each of the four experimental conditions (control, 1.0 THz and 0.1 μJ THz pulses, and UVA). Following exposure, the tissues were incubated at 37° C. in 5% CO₂ atmosphere for 30 minutes in a multiwell dish filled with fresh medium, followed by a snap freeze in liquid nitrogen. Control tissues underwent the same procedure other than being irradiated. The central region (2 mm in diameter, as shown in FIG. 1c) containing the exposed portion of the frozen tissues was cut out and used for gene expression analysis.

### Whole-Genome Gene Expression Profiling.

RNA Isolation.

Total RNA was isolated using the Illustra RNAspin mini kit (GE Healthcare Life Sciences, Buckinghamshire, UK) were processed following the manufacturer's instructions. Samples were eluted in Ultrapure DNase/RNase-free distilled water, which was provided in the kit. RNA samples were quantified using ultraviolet spectroscopy (NanoDrop, Wilmington, Del.) and were further assessed for RNA integrity (RIN) on the Agilent 2100 Bioanalyzer (Santa Clara, Calif.) using the RNA Nano-chip Kit. RNA samples with RIN values of seven or better were used for the further analysis.

Library Preparation.

CRNA was created using the Ambion's Illumina TotalPrep RNA Amplification Kit (Applied Biosystems, Carlsbad, Calif.) with an input of 400 ng of total RNA per sample. Briefly, oligo-dT primers were used to synthesize first strand cDNA containing a phage T7 promoter sequence. Single-stranded cDNA was converted into a double-stranded DNA template via DNA polymerase. RNase H simultaneously acted to degrade the RNA. Samples of cDNA were purified in filter cartridges to remove excess RNA, primers, enzymes and salts. The recovered cDNA was subjected to in vitro transcription using biotinylated UTPs. This step created, labeled and amplified cRNA. A final purification step removed unincorporated NTPs, salts, inorganic phosphates and enzymes, which prepared the samples for hybridization.

Hybridization and Detection.

Illumina's direct hybridization assay kit was used to process samples according to the manufacturer's protocol (Illumina, San Diego, Calif.). Overnight, 750 ng from each cRNA sample was hybridized into the Illumina HumanHT-12_v4 Whole Genome Expression BeadChip arrays. Afterward, a 10-min incubation with a supplied wash buffer at 55° C. preceded a 5-min room temperature (˜22° C.) wash. The arrays were incubated in 100% ethanol for 10 min. A second room temperature wash lasted 2 min with gentle shaking, which completed this high stringency wash step. The arrays were blocked with a buffer for 10 min and washed before a 10-min steptavidin-Cy3 (1:1,000) probing. After a 5-min wash at room temperature, the BeadChips were dried and imaged. Six controls were also built into the Whole-Genome Gene Expression Direct Hybridization Assay system to cover aspects of the array experiments, including controls for: the biological specimen (14 probes for housekeeping controls), 3 controls for hybridization (6 probes for Cy3-labeled hybridization, 4 probes for low stringency hybridization, and 1 probe for high stringency hybridization), signal generation (2 probes for biotin control), and approximately 800 probes for negative controls on an 8-sample BeadChip. The arrays were scanned on the iScan platform (Illumina), and data were normalized and scrutinized using Illumina BeadStudio Software.

BeadChip Statistical Analysis and Data Processing.

The false discovery rate (FDR) was controlled using the Benjamini-Hochberg method. The Illumina Custom Model took the FDR into account and was used to analyze the data. Differential gene expression (at least a 2-fold change) from sham-treated tissues was determined to be statistically significant if the p value after the Benjamini-Hochberg method adjustment was lower than 0.05. The values were transformed to show a log 2 scale. Lists of regulated transcripts were inserted into the web based DAVID Bioinformatics Resources 6.7 (NIAID/NIH) Functional Annotation Tool.

Accession Numbers.

The raw and processed gene expression data for this project have been deposited in the Gene Expression Omnibus (GEO) database, www.ncbi.nlm.nih.gov/geo (GSE48586).

What is claimed is:

1. A method for treating a disease state in skin cells or skin tissue using terahertz radiation, the method comprising the steps of:
a) providing a source of terahertz radiation; and
b) exposing the skin cells or skin tissue to pulses of terahertz radiation in an effective amount to induce modulation of gene expression in the skin cells or skin tissue;
wherein the modulation of gene expression results in a reduction of the disease state in the skin cells or skin tissue.

2. The method of claim 1 wherein the terahertz radiation has a bandwidth of about 0.1 to about 10 terahertz.

3. The method of claim 2 wherein the terahertz radiation has a bandwidth of about 0.2-2.5 terahertz.

4. The method of claim 1 wherein the pulse of terahertz radiation is about 0.2 picoseconds to about 100 picoseconds in duration.

5. The method of claim 4 wherein the pulse of terahertz radiation is about 10 picoseconds in duration.

6. The method of claim 1 wherein the duration of exposure to terahertz radiation is about 1 minute to about 30 minutes.

7. The method of claim 1 wherein the pulses of terahertz radiation have a pulse energy of about 0.01 microjoules to about 100 microjoules.

8. The method of claim 7 wherein the pulses of terahertz radiation have a pulse energy of about 0.1 microjoules to about 2.0 microjoules.

9. The method of claim 1 wherein the pulses of terahertz radiation have a repetition rate of about 1 hertz to about 100 megahertz.

10. The method of claim 9 wherein the pulses of terahertz radiation have a repetition rate of about 1 kilohertz.

11. The method of claim 1 wherein the modulation of gene expression is a down-regulation of gene expression.

12. The method of claim 1 wherein the modulation of gene expression is an up-regulation of gene expression.

13. The method of claim 1 wherein the disease state is skin cancer.

14. The method of claim 13 wherein the skin cancer is non-melanoma skin cancer.

15. The method of claim 14 wherein the non-melanoma skin cancer is a squamous cell carcinoma or basal cell carcinoma.

16. The method of claim 1 wherein the disease state is an inflammatory skin disease.

17. The method of claim 14 wherein the disease state is psoriasis or atopic dermatitis.

18. The method of claim 11 wherein at least one of the genes down-regulated is selected from the group consisting of S100P, S100A11, S100A12, S100A15, SPRR2A, SPRR3, SPRR1B, SPRR2B, SPRR2C, LCE3, LCE3D, CD24, DEFB103A, DEFB4, LOC728454, DEFB1, IVL, SERPINB3, SERPINB4 and SERPINB13.

19. The method of claim 12 wherein at least one of the genes up-regulated is selected from the group consisting of LCE1A, LCE1B, LCE1C, LCE1D, LCE1E, LCE1F and LCE5A.

20. A method for treating a patient with skin cancer using terahertz radiation, the method comprising the steps of:
a) providing a source of terahertz radiation; and
b) exposing the patient to pulses of terahertz radiation, wherein the pulses of terahertz radiation have a bandwidth of about 0.2-2.5 terahertz, a pulse energy of about 0.1-1.0 microjoules for about 30 minutes;
wherein the exposure to the terahertz radiation is an effective amount to induce at least a 1.5 fold change in gene expression in the skin cancer cells as compared to a control sample not exposed to terahertz radiation; and
wherein the change in gene expression results in a reduction in the level of skin cancer in the patient.
